(19) United States
(12) Patent Application Publication
(10) Pub. No.: US 2025/0154259 A1
(43) Pub. Date: May 15, 2025

(54) NOVEL METHODS OF THERAPY

(71) Applicant: BIVICTRIX LIMITED, Macclesfield (GB)

(72) Inventor: Tiffany Thorn, Macclesfield (GB)

(21) Appl. No.: 18/836,975

(22) PCT Filed: Feb. 17, 2023
(86) PCT No.: PCT/GB2023/050368

§ 371 Date: Aug. 8, 2024

(30) Foreign Application Priority Data
Feb. 17, 2022 (GB) 2202171.1

(51) Int. Cl. C07K 16/28; A61K 47/68

(52) U.S. Cl. CPC C07K 16/2827; A61K 47/68031; A61K 47/6851; C07K 16/2803; C07K 2317/31; C07K 2317/622; C07K 2317/732

(57) ABSTRACT

The present invention relates to compositions for use in the treatment of a malignancy, wherein the composition comprises an agent that binds CD33 and B7H4. The present invention also relates to combinations agents targeting the protein pairs.

### TECHNICAL FIELD OF INVENTION

The present invention relates to a composition for use in treatment of a malignancy wherein the composition comprises an agent that binds to CD33 and B7H4. The present invention also relates to combinations of agents that bind to CD33 and B7H4, bispecific antibodies that bind to CD33 and B7H4 and methods for identifying compositions for use in the treatment of malignancies.

### BACKGROUND TO THE INVENTION

One of the many potential side effects of cancer and its treatments is a suppressed immune system, or immunosuppression.

A specific type of immunosuppression, myelosuppression is a condition in which bone marrow activity is decreased, resulting in fewer red blood cells, white blood cells, and platelets. Myelosuppression is often a side effect of some cancer (or other) treatments. Myelosuppression is problematic and potentially very dangerous for patients. Myelosuppression is one of the most common safety concerns in single antigen directed therapies. For example, Gemtuzumab ozogamicin is an immunoconjugate of an anti-CD33 antibody and a toxic calicheamicin-yl derivative. One of the major side-effects of Gemtuzumab ozogamicin includes myelosuppression.

It is an object of the present invention to provide therapies for diseases in which specific cell surface protein pairs, i.e. CD33 and B7H4, are implicated. It is also an object of the invention to provide therapies for diseases in which the specific cell surface protein pairs are implicated which do not result in immunosuppression and/or myelosuppression. It is a further object of the invention to provide methods of determining which specific therapies targeting specific cell surface protein pairs would be successful in preventing immunosuppression and/or myelosuppression and/or impaired immune function. It would be desirable to identify a therapy which can be used for the treatment of a malignancy, such as a haematological cancer (for example Multiple Myeloma or Acute Myeloid Leukaemia (AML)), or a solid tumour cancer (for example a Gynaecological Cancer) which is non-immune suppressing.

### SUMMARY OF THE INVENTION

The inventor has surprisingly shown that targeting both CD33 and B7H4 on tumour-associated immune cells found in the tumour microenvironment provides an effective way of treating cancer as the dual targeting of this specific antigen pair targets tumour associated cells, but not healthy cells.

The invention relates to a composition for use in the treatment of a malignancy, e.g. cancer, wherein the composition comprises an agent that binds to CD33 and B7H4.

The invention also relates to a bispecific antibody or antibody fragment capable of binding CD33 and B7H4 for use in the treatment of a malignancy, e.g. cancer.

The invention also relates to a method for treating a malignancy comprising administering to a subject in need thereof an agent that binds to CD33 and B7H4.

The invention also relates to a method of targeting cells that express both CD33 and B7H4 comprising administering to a subject an agent that binds to CD33 and B7H4.

The invention also relates to a combination of agents for use in the treatment of a malignancy, e.g. cancer, wherein the agents bind to CD33 and B7H4.

The invention also relates to a bispecific antibody or antigen binding fragment thereof capable of binding CD33 and B7H4.

The invention also relates to a pharmaceutical composition comprising an antibody or fragment thereof as described herein.

The invention also relates to a kit comprising an antibody or fragment thereof as described herein.

The following relates to all aspects above:

The treatment may be a non-immune suppressing treatment.

The non-immune suppressing treatment may be non-myelosuppressing treatment.

The agent may be an antibody or antigen binding fragment thereof.

The agent may be a bispecific antibody or antigen binding fragment thereof that binds CD33 and B7H4.

The composition may further comprise a payload.

The payload may be a cell killing agent, an immune-modulating payload, a macrophage class switching agent, or a light activatable payload.

The immune-modulating payload may be a STING agonist or a toll-like receptor agonist.

The cell killing agent may comprise a cytotoxin.

The cytotoxin may be selected from:
i) a peptide toxin; or
ii) a chemical toxin.

The composition may further comprise a linker for linking the payload to the agent that binds to CD33 and B7H4 expressed on the cell surface.

The composition may be a bispecific antibody drug conjugate (ADC).

The malignancy may comprise a malignant disease associated with an increase in myeloid derived suppressor cells.

The malignancy may be an ovarian cancer, an ovarian cancer derived cancer, Acute Myeloid Leukaemia (AML) or AML derived cancer.

The malignancy may be selected from one of the following cancers: haematological cancers including Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukaemia (CLL), Myelodysplastic Syndrome (MDS), and Diffuse Large B cell Lymphoma (DLBCL); or Hepatocellular Carcinoma; or Gynaecological Cancers including Ovarian Cancer, Cervical Cancer, and Endometrial Cancer; lung cancer, bladder cancer, breast cancer, renal cell carcinoma, melanoma, colorectal cancer, gastric cancer, pancreatic cancer, thyroid cancer, liver cancer and glioblastoma.

### DETAILED DESCRIPTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols 1 and 2, Ontermann and Dubel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients. Suitable assays to measure the properties of the molecules disclosed herein are also described in the examples.

In accordance with a first aspect of the present invention, there is provided a composition for use in the treatment of a malignancy, wherein the composition comprises an agent, for example a cell inhibiting agent, that binds to CD33 and B7H4.

The composition may be provided for use in the non-immune suppressing and/or non-myelosuppressing treatment of a malignancy, wherein the composition comprises an agent, for example a cell inhibiting agent, that binds to CD33 and B7H4.

The inventor has evaluated co-expression of antigens on AML cells and healthy cells. The inventor has surprisingly found that tumour-associated immune cells express both CD33 and B7H4 antigens on their cell surface whilst healthy cells do not express both antigens. This makes it possible to selectively target tumour associated cells.

Thus, unexpectedly, and advantageously, the present inventor has identified that cells, e.g. tumour-associated immune cells, presenting both CD33 and B7H4 can be targeted with one or more agents which can bind both proteins without any or reduced myelosuppression or immune suppression. In contrast, the inventor showed that agents targeting malignant cells expressing other antigen pairs, e.g. CD25 and CD34; or CD56 and CD7; or CD56 and CD11c; or CD33 and CD371 were not suitable as treatments for targeting malignant cells expressing those antigens as such targeting would result in immune suppression and/or myelosuppression and/or impaired immune function. This is because these target pairs were not only expressed on malignant cells, but also on healthy cells.

These results presented herein show that targeting CD33 and B7H4 with a bispecific antibody or antigen binding fragment thereof targets tumour-associated immune cells found in the tumour microenvironment expressing both CD33 and B7H4 while avoids targeting healthy haematological cell populations. Such cells include myeloid derived suppressor cells (MDSCs).

Therefore, targeting both CD33 and B7H4 surprisingly avoids or reduces off-target cytotoxic effects. Off-target cytotoxicity that targets healthy cells, e.g. PBMC and/or BMMC, leads to immune suppression and/or myelosuppression and/or impaired immune function which are common side effects of anti-cancer chemotherapy. Targeting both CD33 and B7H4 antigens which are expressed on the surface of tumour-associated immune cells but are not expressed on healthy cells, e.g. haematological cells, therefore avoids or reduces such off-target cytotoxicity of these healthy cells. This reduces immune suppression and/or myelosuppression and/or impairment of immune function.

Targeting the cells and treating cancer can be achieved with agents as described herein, for example bispecific antibodies antigen binding fragments thereof.

The composition of the invention may be or comprise an antibody or antigen binding fragment thereof. Thus, in one embodiment the agent comprises or consists of an antibody or antigen binding fragment thereof capable of binding to both CD33 and B7H4.

The composition may further comprise a payload, for example a cell killing agent, an immune-modulating payload, a macrophage class switching agent or a light activatable payload. In certain embodiments, the cell killing agent comprises a cytotoxin. The cytotoxin may be selected from: i) a peptide toxin; or ii) a chemical toxin. The cytotoxin may be selected from: i) a peptide toxin ii) a chemical toxin, iii) an inhibitor of Bcl-2 or Bcl-axl, iv) an RNA Polymerase inhibitor such as α-amanitin, v) a spliceosome inhibitor, vi) a microtubule-targeting payload, or vii) a DNA-damaging payload. The skilled addressee will appreciate that a range of toxins will be compatible with the composition. Preferably the cell killing agent is auristatin MMAF. Suitable toxins are further exemplified herein.

The composition may further comprise a linker for linking the payload, e.g. cell killing agent, to the agent, e.g. cell inhibiting agent, and/or antibody or antibody fragment, that binds to CD33 and B7H4 expressed on the cell surface, e.g. a bispecific antibody or antibody fragment. Preferably the linker is a non-cleavable maleimidoca-proyl (mc) linker. Suitable linkers are further exemplified herein.

Thus, the agent may be or comprise an antibody drug conjugate (ADC).

In certain embodiments, the composition comprises a multispecific, e.g. bispecific, antibody drug conjugate. In an embodiment, the composition comprises or consists of a bispecific antibody or antibody fragment drug conjugate. In another embodiment, the composition is a trispecific antibody drug conjugate. In such an embodiment, the antibody or antigen binding fragment thereof binds CD33, B7H4 and a further antigen target. For example, a half life extending moiety may be included which binds human serum albumin.

The skilled addressee will appreciate that the composition could be used for treating a number of diseases in which CD33 and B7H4 are implicated. The disease in which CD33 and B7H4 cell surface protein pairs are implicated may comprise a malignant disease associated with an increase in myeloid derived suppressor cells. The disease in which CD33 and B7H4 are implicated may be a malignant cancer associated with an increase in myeloid derived suppressor cells. The malignant cancer may be selected from one of the following: haematological cancers (such as Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukaemia (CLL), Myelodysplastic Syndrome (MDS) and Diffuse Large B cell Lymphoma (DLBCL)), Hepatocellular Carcinoma, Gynaecological Cancers (such as Ovarian Cancer, Cervical Cancer and Endometrial Cancer), lung cancer, bladder cancer, breast cancer, renal cell carcinoma, melanoma, colorectal cancer, gastric cancer, pancreatic cancer, thyroid cancer, liver cancer and glioblastoma.

In accordance with a second aspect of the present invention, there is provided a combination of agents, e.g. cell inhibiting agents, for example, for use in the treatment of a malignancy, wherein the cell inhibiting agents bind CD33 and B7H4.

The combination of agents may be provided for use in the non-immune suppressing and/or non-myelosuppressing treatment of a malignancy, wherein the agents bind CD33 and B7H4.

The agents, e.g. cell inhibiting agents, may comprise antibodies or antigen binding fragments thereof. In certain embodiments, the cell inhibiting agent may further comprise a payload, for example a cell killing agent, an immune-modulating payload, macrophage class switching agent or a light activatable payload. The cell killing agent may comprise a cytotoxin. The cytotoxin may be selected from: i) a peptide toxin; or ii) a chemical toxin. The cytotoxin may be selected from: i) a peptide toxin ii) a chemical toxin, iii) an inhibitor of Bcl-2 or Bcl-axl, iv) an RNA Polymerase inhibitor such as α-amanitin, v) a spliceosome inhibitor, vi) a microtubule-targeting payload, or vii) a DNA-damaging payload. Preferably the cell killing agent is auristatin MMAF. The skilled addressee will appreciate that a range of toxins will be compatible with the agents. Suitable toxins are further exemplified herein.

The agents may further comprise a linker for linking the payload, e.g. cell killing agent, to the agent that binds to at least one of the pair of proteins expressed on the cell surface, e.g. a bispecific antibody or antibody fragment that binds CD33 and B7H4. Preferably the linker is a non-cleavable maleimidoca-proyl (mc) linker. Suitable linkers are further exemplified herein.

Thus, the agent may be an antibody drug conjugate.

The disease in which CD33 and B7H4 are implicated may comprise a malignant disease associated with an increase in myeloid derived suppressor cells. The disease in which CD33 and B7H4 are implicated may be a malignant cancer. The disease in which CD33 and B7H4 cell surface protein pairs are implicated may be an ovarian cancer or an ovarian cancer derived cancer or an acute myeloid leukaemia (AML) or an AML derived cancer. The cancer may be selected from one of the following: haematological cancers (such as Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukaemia (CLL), Myelodysplastic Syndrome (MDS) and Diffuse Large B cell Lymphoma (DLBCL)), Hepatocellular Carcinoma, Gynaecological Cancers (such as Ovarian Cancer, Cervical Cancer and Endometrial Cancer), lung cancer, bladder cancer, breast cancer, renal cell carcinoma, melanoma, colorectal cancer, gastric cancer, pancreatic cancer, thyroid cancer, liver cancer and glioblastoma.

In accordance with a third aspect of the present invention, there is provided an agent, for example a cell inhibiting agent, for use in the treatment of a malignancy, wherein the cell inhibiting agent bispecifically binds to CD33 and B7H4.

The agent may be provided for use in the non-immune suppressing and/or non-myelosuppressing treatment of a malignancy, wherein the cell inhibiting agent bispecifically binds CD33 and B7H4.

The agent may be an antibody or antigen binding fragment thereof. The cell inhibiting agent further comprises a payload, for example a cell killing agent, an immune-modulating payload, macrophage class switching agent or a light activatable payload.

The agents may comprise antibodies or antigen binding fragments thereof. In certain embodiments, the cell inhibiting agent may further comprise a payload, for example a cell killing agent, an immune-modulating payload, a macrophage class switching agent or a light activatable payload. The cell killing agent may comprise a cytotoxin. The cytotoxin may be selected from: i) a peptide toxin; or ii) a chemical toxin. The cytotoxin may be selected from: i) a peptide toxin ii) a chemical toxin, iii) an inhibitor of Bcl-2 or Bcl-axl, iv) an RNA Polymerase inhibitor such as α-amanitin, v) a spliceosome inhibitor, vi) a microtubule-targeting payload, or vii) a DNA-damaging payload. The skilled addressee will appreciate that a range of toxins will be compatible with the agents. Preferably the cell killing agent is auristatin MMAF. Suitable toxins are further exemplified herein.

The agents may further comprise a linker for linking the payload, e.g. cell killing agent, to the agent that binds to at least one of the pair of proteins expressed on the cell surface. Preferably the linker is a non-cleavable maleimidoca-proyl (mc) linker. Suitable linkers are further exemplified herein.

The agent may be an antibody drug conjugate.

The disease in which CD33 and B7H4 are implicated may comprise a malignant disease associated with an increase in myeloid derived suppressor cells. The disease in which CD33 and B7H4 are implicated may be a malignant cancer. The disease in which CD33 and B7H4 are implicated may be an ovarian cancer or an ovarian cancer derived cancer or an acute myeloid leukaemia (AML) or an AML derived cancer. The cancer may be selected from one of the following: haematological cancers (such as Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukaemia (CLL), Myelodysplastic Syndrome (MDS) and Diffuse Large B cell Lymphoma (DLBCL)), Hepatocellular Carcinoma, Gynaecological Cancers (such as Ovarian Cancer, Cervical Cancer and Endometrial Cancer), lung cancer, bladder cancer, breast cancer, renal cell carcinoma, melanoma, colorectal cancer, gastric cancer, pancreatic cancer, thyroid cancer, liver cancer and glioblastoma.

The antibody or antigen binding fragment thereof, or cell inhibiting agent, as herein above described with reference to all aspects may be for use in the treatment of a CD33+B7H4+ malignancy, such as a haematological malignancy or solid tumour malignancy including ovarian cancer, lung cancer, bladder cancer, breast cancer, renal cell carcinoma, melanoma, colorectal cancer, gastric cancer, pancreatic cancer, thyroid cancer, liver cancer and glioblastoma.

In another aspect, the invention provides a bispecific antibody or antigen binding fragment thereof that is capable of binding CD33 and B7H4 for use in the treatment of cancer. Also provided is a method of treating cancer comprising administering to a subject in need thereof a bispecific antibody or antigen binding fragment thereof capable of binding CD33 and B7H4. As explained herein, the antibody or antigen binding fragment thereof may be linked to a payload, for example a cell killing agent, an immune-modulating payload, a macrophage class switching agent or a light activatable payload.

In another aspect, the invention provides a bispecific antibody or antigen binding fragment thereof capable of binding CD33 and B7H4. As explained herein, bispecific antibody may linked to a payload, for example a cell killing agent, an immune-modulating payload, a macrophage class switching agent or a light activatable payload.

The invention also provides a nucleic acid encoding a bispecific antibody or fragment thereof as described herein.

The invention also provides a host cell expressing a nucleic acid encoding a bispecific antibody or fragment thereof as described herein. The host cell may be a bacterial, viral, insect, plant, mammalian or other suitable host cell. In one embodiment, the cell is an E. coli cell. In another embodiment, the cell is a yeast cell. In another embodiment, the cell is a Chinese Hamster Ovary (CHO) cell.

The term bispecific antibody refers to a binding molecule which binds to two different antigens, i.e. CD33 and B7H4.

In another aspect, the invention provides a pharmaceutical composition comprising a bispecific antibody or fragment thereof capable of binding CD33 and B7H4.

In another aspect, the invention provides a kit comprising a bispecific antibody or fragment thereof capable of binding CD33 and B7H4 and optionally instructions for use of said kit.

In another aspect, the invention provides an in vivo, in vitro or ex vivo method of targeting cells that express both CD33 and B7H4 comprising administering to a subject an agent, e.g. a bispecific antibody or fragment thereof, that binds to CD33 and B7H4.

In another aspect, the invention provides a method for reducing off target toxicity of a cancer treatment comprising administering to a subject an agent, e.g. a bispecific antibody or fragment thereof, that binds to CD33 and B7H4.

The antibody or antigen binding fragments thereof, or agents, e.g. cell inhibiting agents, as herein above described with reference to all aspects may be for use in the treatment of a CD33+B7H4+ malignancy, such as a haematological malignancy and/or a cancer.

The antibody or antigen binding fragments thereof, or agents, e.g. cell inhibiting agents, as herein above described with reference to all aspects may be for a method of treating a malignancy in an individual in need therefore, e.g. a cancer where the method comprises administering the antibody or antigen binding fragments thereof. Thus, the invention also relates to a method of treating cancer comprising administering to an individual in need thereof an agent, e.g. a bispecific antibody or fragment thereof, that binds to CD33 and B7H4.

According to the various aspects of the invention, it is preferred that the antibody or antigen binding fragments thereof, or agents, e.g. cell inhibiting agents, thereof are artificially generated. According to the various aspects of the invention, the antibody or antigen binding fragments thereof, or agents, e.g. cell inhibiting agents are isolated.

The term “isolated” refers to a moiety that is isolated from its natural environment. For example, the term “isolated” refers to an antibody, that is substantially free of other antibodies or binding molecules. Moreover, an isolated antibody or antigen binding fragment thereof may be substantially free of other cellular material and/or chemicals.

In another related aspect of the present invention, there is provided an antibody or antigen binding fragments thereof, or agent, e.g. cell inhibiting agent, as herein above described with reference to all aspects for use in the manufacture of a medicament for a malignancy.

As used herein “a medicament” refers to a substance used for medical treatment (i.e., a medicine). The medicament may be, e.g., a T cell product that is for use in adoptive cell transfer.

As used herein CD33 and B7H4 is preferably human CD33 and B7H4. In certain embodiments, the agents, bispecific antibodies or antigen binding fragments thereof specifically bind to CD33 and B7H4 that are cell surface expressed. As used herein, the expression “cell surface-expressed” means CD33 and B7H4 proteins that are expressed on the surface of a cell in vitro or in vivo, such that at least a portion of a CD33 and/or B7H4 protein is exposed to the extracellular side of the cell membrane and is accessible to the agent, bispecific antibody or antigen binding fragment thereof of the invention. CD33 and B7H4 are found on tumour-associated immune cells, specifically myeloid derived suppressor cells (MDSCs).

The term “malignancy” or “disease” as used herein refers to a malignancy characterised by the expression of both CD33 and B7H4 proteins on the surface of myeloid derived suppressor cells associated with a malignant disease (e.g., a malignancy associated with an increase in myeloid derived suppressor cells that express CD33 and/or B7H4 protein at levels considered acceptable for therapy with the antibody or antigen binding fragments thereof, or agents, e.g. cell inhibiting agents, that specifically binds to CD33 and B7H4 protein).

In one example for all embodiments of the invention, the term malignancy refers to malignant indications that are showed to have an associated with MDSCs. In another example, the term refers to a disease characterised by a myeloid derived suppressor cell surface or myeloid derived suppressor cell protein expression pattern as described above. Such a cell type may be any myeloid derived suppressor cell type of the human body and in particular a malignancy associated with increased myeloid derived suppressor cells, such as haematological cancers (such as Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukaemia (CLL), Myelodysplastic Syndrome (MDS) and Diffuse Large B cell Lymphoma (DLBCL)), Hepatocellular Carcinoma, Gynaecological Cancers (such as Ovarian Cancer, Cervical Cancer and Endometrial Cancer), lung cancer, bladder cancer, breast cancer, renal cell carcinoma, melanoma, colorectal cancer, gastric cancer, pancreatic cancer, thyroid cancer, liver cancer and glioblastoma.

The specific cells targeted by CD33×B7H4 may be myeloid derived suppressor cells within multiple solid and haematological indications, such as Gynaecological Cancers including Ovarian Cancer, Cervical Cancer and Endometrial Cancer; Haematological Cancers including AML, Multiple Myeloma, T-cell Acute Lymphoblastic Leukaemia, Blastic Plasmacytoid Dendritic Cell Neoplasm, Chronic Lymphocytic Leukaemia, Myelodysplastic Syndrome and Diffuse Large B Cell Lymphoma; Hepatocellular Cancers, lung cancer, bladder cancer, breast cancer, renal cell carcinoma, melanoma, colorectal cancer, gastric cancer, pancreatic cancer, thyroid cancer, liver cancer and glioblastoma.

According to one embodiment of the various aspects of the invention, the cancer may be selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, breast cancer, brain cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, lung cancer, non-small cell lung cancer, thymoma, urothelial carcinoma leukemia, prostate cancer, mesothelioma, adrenocortical carcinoma, lymphomas, such as such as Hodgkin's disease, non-Hodgkin's, gastric cancer, Chronic myeloid leukaemia, Myelodysplastic syndromes and multiple myelomas.

In one embodiment of the various aspects of the invention, the tumour is a solid tumour. Examples of solid tumours which may be accordingly treated include breast carcinoma, lung carcinoma, colorectal carcinoma, pancreatic carcinoma, glioma and lymphoma. Some examples of such tumours include epidermoid tumours, squamous tumours, such as head and neck tumours, colorectal tumours, prostate tumours, breast tumours, lung tumours, including small cell and non-small cell lung tumours, pancreatic tumours, thyroid tumours, ovarian tumours, and liver tumours. Other examples include Kaposi's sarcoma, CNS, neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma. Examples of vascularized skin cancers include squamous cell carcinoma, basal cell carcinoma and skin cancers that can be treated by suppressing the growth of malignant keratinocytes, such as human malignant keratinocytes.

In one embodiment of the various aspects of the invention, the tumour is a non-solid tumour. Examples of non-solid tumours include leukemia, multiple myeloma and lymphoma.

It will be apparent to the skilled person that each protein may have a number of alternative names by which each protein is known. For example (non-exhaustive): CD33 may be known as: Siglec-3, sialic acid binding Ig-like lectin 3, SIGLEC3, SIGLEC-3, gp67, p67; and B7H4 may be known as: VTCN1, B7-H4, B7S1, B7X, B7h.5, PRO1291, V-set domain containing T cell activation inhibitor 1.

CD33 is a 67 kDa plasma membrane protein that binds to sialic acid and is a member of the sialic acid-binding Ig-related lectin (SIGLEC) family of proteins. CD33 is known to be expressed on myeloid cells. CD33 expression has also been reported on a number of malignant cells.

Wild type human CD33 has been described, see e.g. UniProt Accession No. P20138).

An amino acid sequence for CD33 is shown below (SEQ ID No. 1).

MPLLLLLPLLWAGALAMDPNFWLQVQESVTVQEGLCVLVPCTFFHPIPY
YDKNSPVHGYWFREGAIISGDSPVATNKLDQEVQEETQGRFRLLGDPSR
NNCSLSIVDARRRDNGSYFFRMERGSTKYSYKSPQLSVHVTDLTHRPKI
LIPGTLEPGHSKNLTCSVSWACEQGTPPIFSWLSAAPTSLGPRTTHSSV
LIITPRPQDHGTNLTCQVKFAGAGVTTERTIQLNVTYVPQNPTTGIFPG
DGSGKQETRAGVVHGAIGGAGVTALLALCLCLIFFIVKTHRRKAARTAV
GRNDTHPTTGSASPKHQKKSKLHGPTETSSCSGAAPTVEMDEELHYASL
NFHGMNPSKDTSTEYSEVRTQ

Thus, the agent, e.g. bispecific antibody or antibody fragment thereof, according to the various aspects of the invention binds to SEQ ID NO. 1 or a variant thereof. A variant has at least 80%, 85%, 90% or 95% sequence identity to SEQ D NO. 1.

CD33 plays a role in the regulation of cellular calcium influx necessary for the development, differentiation, and activation of B-lymphocytes.

Unless otherwise specified, the term CD33 as used herein refers to human CD33. CD33 is also known as “Membrane Spanning 4-Domains A1”, “Bp35” or “FMC7”, and these terms are used interchangeably and include variants and isoforms of human CD33.

The terms “CD33 binding molecule/protein/polypeptide/agent/moiety”, “CD33 antigen binding molecule molecule/protein/polypeptide/agent/moiety”, “anti-CD33 antibody”, “anti-CD33 antibody fragment”, “anti-CD33 antibody or antigen binding portion thereof” or “CD33 antibody” all refer to a molecule capable of specifically binding to the human CD33 antigen.

B7H4, also known as V-Set Domain Containing T Cell Activation Inhibitor 1 or VTCN1, belongs to the B7 costimulatory protein family. Proteins in this family are present on the surface of antigen-presenting cells and interact with ligand bound to receptors on the surface of T cells. B7H4 is a type I transmembrane protein that includes a short intracellular domain, a hydrophobic transmembrane domain, and an extracellular domain with an IgV- and an IgC-like domain with four conserved cysteine residues and seven sites for N-linked glycosylation. Studies have shown that high levels of the encoded protein correlated with tumour progression. In cancer, B7H4 expression is correlated with advanced stages of cancer, poor prognosis, and decreased overall patient survival. Unless otherwise stated, the term B7H4 terms refers to human B7H4 and includes variants and isoforms of human B7H4. The amino acid sequence of human B7-H4 may have Genbank accession number NP 078902 (version 2).

An amino acid sequence for B7H4 is shown below (SEQ ID No. 2).

(SEQ ID NO. 2)
MASLGQILFWSIISIIIILAGAIALIIGFGISGRHSITVTTVASAGNIG
EDGILSCTFEPDIKLSDIVIQWLKEGVLGLVHEFKEGKDELSEQDEMFR
GRTAVFADQVIVGNASLRLKNVQLTDAGTYKCYIITSKGKGNANLEYKT
GAFSMPEVNVDYNASSETLRCEAPRWFPQPTVVWASQVDQGANFSEVSN
TSFELNSENVTMKVVSVLYNVTINNTYSCMIENDIAKATGDIKVTESEI
KRRSHLQLLNSKASLCVSSFFAISWALLPLSPYLMLK

Thus, the agent, e.g. bispecific antibody or antibody fragment thereof, according to the various aspects of the invention binds to SEQ ID NO. 2 or a variant thereof. A variant has at least 80%, 85%, 90% or 95% sequence identity to SEQ D NO. 2.

The terms “B7H4 binding molecule/protein/polypeptide/agent/moiety”, “B7H4 antigen binding molecule molecule/protein/polypeptide/agent/moiety”, “anti-B7H4 antibody”, “anti-B7H4 antibody fragment”, “anti-B7H4 antibody or antigen binding portion thereof” or “B7H4 antibody” all refer to a molecule capable of specifically binding to the human B7H4 antigen.

The terms “antigen(s)” and “epitope(s)” are well established in the art and refer to the portion of a protein or polypeptide which is specifically recognized by a component of the immune system, e.g. an antibody or a T-cell/B-cell antigen receptor. As used herein, the term “antigen(s)” encompasses antigenic epitopes, e.g. fragments of antigens which are recognized by, and bind to, immune components. Epitopes can be recognized by antibodies in solution, e.g. free from other molecules. Epitopes can also be recognized by T-cell antigen receptors when the epitope is associated with a class I or class II major histocompatibility complex molecule.

The term “epitope” or “antigenic determinant” refers to a site on the surface of an antigen to which an immunoglobulin, antibody or antigen-binding fragment thereof specifically binds. Generally, an antigen has several or many different epitopes and reacts with many different antibodies. The term “specifically” includes linear epitopes and conformational epitopes.

Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antigen-binding fragment thereof (i.e., epitope mapping) are well known in the art and include, for example, immunoblotting and immunoprecipitation assays, wherein overlapping or contiguous peptides are tested for reactivity with a given antibody or antigen-binding fragment thereof. Competition assays can also be used to determine if a test antibody binds to the same epitope as a reference antibody. Suitable competition assays are mentioned elsewhere herein and also shown in the examples. In some aspects, the epitope to which an antibody or antigen-binding fragment thereof binds can be determined by, e.g, NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g, site-directed mutagenesis mapping).

The term “binding molecule” “antibody” or “antigen binding molecule” as used herein refers to an immunoglobulin protein that is capable of binding an antigen target of interest, i.e. CD33 and B7H4. In particular, the term “antibody” as used herein broadly refers to any polypeptide comprising complementarity determining regions (CDRs) that confer specific binding affinity of the polypeptide for an antigen. The term antibody as used herein encompasses polyclonal and monoclonal antibody preparations. The term “antibody” as used herein encompasses binding molecules with different antibody formats as well as antigen binding fragments of antibodies.

The antibody or antigen-binding fragment thereof described herein, “which binds” or is “capable of binding” the antigen of interest, binds the antigen with sufficient affinity such that the antibody or antigen-binding fragment thereof is useful as a therapeutic or diagnostic agent in targeting CD33 and B7H4 as described herein. The term “specific” may refer to the situation in which the antibody molecule will not show any significant binding to molecules other than its specific binding partner.

The terms “polypeptide(s)” and “protein(s)” are used interchangeably throughout the application and denote at least two covalently attached amino acids, thus may signify proteins, polypeptides, oligopeptides, peptides, and fragments thereof. The protein may be made up of naturally occurring amino acids and peptide bonds, or synthetic peptidomimetic structures.

Hence, “amino acid(s)” or “peptide residue(s)”, as used herein, denote both naturally occurring and synthetic amino acids. In some cases, the immunoglobulin proteins of the present invention may be synthesized using any in vivo or in vitro protein synthesis technique known in the art.

The antibody or antigen binding fragments thereof, or agents, e.g. cell inhibiting agents, may be capable of inducing CD33 and/or B7H4 receptor mediated internalisation into a CD33+ and/or B7H4+ cell.

CD33+B7H4+ malignancies include, but are not limited to, haematological cancers (such as Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukaemia (CLL), Myelodysplastic Syndrome (MDS) and Diffuse Large B cell Lymphoma (DLBCL)), Hepatocellular Carcinoma, Gynaecological Cancers (such as Ovarian Cancer, Cervical Cancer and Endometrial Cancer), lung cancer, bladder cancer, breast cancer, renal cell carcinoma, melanoma, colorectal cancer, gastric cancer, pancreatic cancer, thyroid cancer, liver cancer and glioblastoma. Other cancers are mentioned elsewhere herein.

The antibody or antigen binding fragments thereof, or agents, e.g. cell inhibiting agents, may bispecifically bind CD33 and B7H4 and wherein the CD33+ and B7H4+ cell is a myeloid derived suppressor cell associated with a malignancy.

The antibody or antigen binding fragments thereof, or agents, e.g. cell inhibiting agents, may be capable of mediating antibody dependent cellular cytotoxicity.

The antibody or antigen binding fragment thereof, or agents, e.g. cell inhibiting agents, may be attached to, or formed with an immune effector cell. The immune effector cell may comprise a T cell and/or a NK cell. Preferably, immune effector cell is a T cell. The immune effector cell may be a bispecific anti-CD33 anti-B7H4 CAR-T. The T cell may comprise a CD33+ T cell, a B7H4+ T cell or a combination thereof. Thus, the agent may be a CAR-T cell.

The antibody or antigen binding fragment thereof, or agents, e.g. cell inhibiting agents may be a trispecific immune cell engager. The antibody or antigen binding fragment thereof, or cell inhibiting agents may additionally comprise an immune cell binding domain. The immune cell binding domain is able to attach to, or bind to, an immune cell. The immune cell binding domain may bind to one or more T cells or NK cells. The immune cell binding domain binds to or attaches to an immune cell causing the immune cell to kill the malignant cell to which the antibody, antigen binding fragment thereof or cell inhibiting agent is bound.

The composition, antibody drug conjugate antibody or antigen binding fragments thereof, or agents, e.g. cell inhibiting agents, may comprise or consist of: i) a payload, for example a cell killing agent, an immune-modulating payload or a light activatable payload; ii) a CD33 binding portion; and iii) a B7H4 binding portion. The CD33 binding portion may be an antibody or antibody fragment thereof. The B7H4 binding portion may be an antibody or antibody fragment thereof.

In alternative embodiments, the CD33 and/or B7H4 binding portion comprises an antigen binding fragment of an antibody, or individual cell inhibiting agents.

An antibody drug conjugate according to the various aspects of the invention shows preferential cytotoxicity for cells expressing both CD33 and B7H4 over cells that express neither of these targets or cells that express only one of these targets.

An antibody drug conjugate according to the invention shows reduced off target cytotoxicity in a CD34+ to CD33+ myeloid differentiation colony forming assay compared to a conjugated antibody that targets a single antigen. The assay may be performed as shown in the examples. An antibody drug conjugate according to the invention may provide selective cell killing of dual positive CD33+B7H4+ cells, e.g. in vivo or in a cytotoxicity assay as shown in the examples.

A bispecific antibody or fragment thereof as described herein is capable of binding both CD33 and B7H4 and is also capable of mediating selective cytotoxicity.

The agent, antibody or antigen binding fragment thereof may be linked or conjugated to a payload. A skilled persons would know that different payloads for such conjugates are known in the art (see e.g. Gingrich J. How the Next Generation Antibody Drug Conjugates Expands Beyond Cytotoxic Payloads for Cancer Therapy-J. ADC. Apr. 7, 2020).

As used in the various aspects and embodiments herein, the payload may be a cell killing agent or cell killing portion. The terms cell killing agent and cell killing portion are used interchangeably.

In one embodiment, the payload may be an immune-modulating payload.

In one embodiment, the payload may be light activatable payload.

As used herein an immune-modulating payload includes any moiety that modulates the immune system, for example which stimulates the immune system and/or kills the target cell. Thus, a moiety that has immuno-activating and/or antineoplastic activities can be used. Such moieties may be synthetic peptides that recognise the specific target and trigger (agonist) or block (antagonist) inflammatory responses. The target may be a pattern recognition receptor (PRR), including Toll-like receptors (TLRs), NOD-like receptors (NLRs), RIG-I-like receptors (RLRs), C-type lectin receptors (CLRs) and cytosolic dsDNA sensors (CDSs).

Examples of payloads include agonists for the stimulator of interferon genes protein (STING; transmembrane protein 173; TMEM173). Such payloads include cyclic dinucleotides and compounds listed in see WO2021113679). Activation of the STING pathway triggers an immune response that results in generation of specific killer T-cells that shrink tumours and can provide long-lasting immunity so the tumours do not recur. Alternatively, payloads that act on toll-like receptors (TLRs) may be used. For example, agonists that bind to TLR7 and/or TLR8 can be used.

Another example of a payload is a macrophage class switching agent.

A light activatable payload (IRDye® 700DX, IR700) may also be used. Light activation of the non-toxic payload results in the generation of singlet oxygen species that damage the cell membrane integrity, resulting in necrotic and immunogenic cell death of tumour cells, resulting in minimal damage to surrounding normal tissue.

The cell killing portion may be a cytotoxin and the skilled addressee will understand that a range of cytotoxins will be compatible with the composition. A cytotoxin may be selected from: i) a peptide toxin, or ii) a chemical toxin, or iii) an inhibitor of Bcl-2 or Bcl-axl, iv) an RNA Polymerase inhibitor such as α-amanitin, v) a spliceosome inhibitor, vi) a microtubule-targeting payload, or vii) a DNA-damaging payload. Preferably the cell killing agent is auristatin MMAF. Other toxins are listed elsewhere herein.

The antibody or antigen binding fragments thereof, or agent, e.g. cell inhibiting agent, may further comprise a linking portion linking the cell kill portion with the CD33 binding portion and/or the B7H4 binding portion. The antibody or antigen binding fragments thereof, or cell inhibiting agents, may be in the format of an antibody drug conjugate.

In the embodiment of a bispecific antibody, then such an antibody may be a full-length antibody. As used herein, the terms “treat”, “treating” and “treatment” are taken to include an intervention performed with the intention of preventing the development or altering the pathology of a disorder or symptom. Accordingly, “treatment” refers to both therapeutic treatment and prophylactic or preventative measures, wherein the object is to prevent or slow down (lessen) the targeted disorder or symptom. Accordingly, the term “treating” encompasses treating and/or preventing the development of a disorder or symptom. As used herein, “therapy” refers to the prevention or treatment of a disease or disorder. Therapy may be prophylactic or therapeutic.

In such aspects, the antibody or antigen binding fragments thereof, or cell inhibiting agents, of the invention are administered to a patient in remission from the malignancy, resulting in preventing or delaying recurrence of the underlying malignancy.

As used herein, a “patient”, “subject” or “individual” is typically a human who is undergoing treatment for, or has been diagnosed as having, malignancy, preferably a CD33+B7H4+ malignancy, e.g. a cancer. In some embodiments, the antibody or antigen binding fragments thereof, or cell inhibiting agents, are administered to a patient in remission from a CD33+B7H4+ malignancy, whereby the recurrence of the malignancy is prevented or delayed. In some embodiments, the patient lacks detectable cells of the malignancy. As used herein, a “lack of detectable cells” is determined by standard diagnostic or prognostic methods. A patient in remission from AML typically exhibits resolution of abnormal clinical features, return to normal blood counts and normal haematopoiesis in the bone marrow with <5% blast cells, a neutrophil count of >1.000-1,500, a platelet count of >100,000, and disappearance of the leukemic clone. See, e.g., The Merck Manual, Sec. 11, Ch. 138 (17th ed. 1997): Estey, 2001, Cancer 92 (5): 1059-1073.

In some embodiments, the patient in remission from the CD33+B7H4+ malignancy has not undergone a bone marrow transplant. In other embodiments, the patient in remission from the CD33+B7H4+ malignancy has undergone a bone marrow transplant. The bone marrow transplant can be either an autologous or an allogeneic bone marrow transplant.

In embodiments treating a CD33+B7H4+ malignancy (for example AML) and delaying preventing or delaying recurrence of CD33+B7H4+ malignancy (for example AML) involves the inducing cancer cell death and/or inhibiting or reducing cancer cell growth.

As used in the various aspects and embodiments herein, the term “reduce” includes reduction by at least 10%, 20%, 30%, 40%, 50%, 60%, 70%, 80%, 90% or more.

The antibody or antigen binding fragments thereof, or agent, e.g. cell inhibiting agent, may be part of a composition (e.g., a therapeutic composition) that comprises the compound (i.e., the antibody or antigen binding fragments thereof, or agents, e.g. cell inhibiting agents) and one or more other components. A composition may be a therapeutic composition that comprises the antibody or antigen binding fragments thereof, or agent, e.g. cell inhibiting agent, and a pharmaceutically acceptable excipient, adjuvant, diluent and/or carrier. Therapeutic compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected compound without causing any undesirable biological effects or interacting in a deleterious manner with any of the other components of the pharmaceutical composition in which it is contained.

Excipients are natural or synthetic substances formulated alongside an active ingredient (e.g., the vaccine, cell cycle inhibitor, modulator of an immune suppression mechanism, or immune check point inhibitor (as appropriate)), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

As used herein, the terms “effective amount” and “therapeutically effective amount” refer to the quantity of the active therapeutic agent sufficient to yield a desired therapeutic response without undue adverse side effects such as toxicity, irritation, or allergic response. The specific “effective amount” will, obviously, vary with such factors as the particular condition being treated, the physical condition of the patient, the type of animal being treated, the duration of the treatment, the nature of concurrent therapy (if any), and the specific formulations employed and the structure of the compounds or its derivatives. In this case, an amount would be deemed therapeutically effective if it resulted in one or more of, but not limited to, the following: (a) the inhibition of cancer cell growth (e.g., AML cells); and (b) the killing of cancer cells (e.g., AML cells).

The dose of the antibody or antigen binding fragments thereof, or agents, e.g. cell inhibiting agents, and therapeutic compositions thereof administered to a patient may vary depending upon the age and the size of the patient, target disease, conditions, route of administration, and the like. The preferred dose is typically calculated according to body weight or body surface area.

Methods of administration of the antibody or antigen binding fragments thereof, or agents, e.g. cell inhibiting agents, and therapeutic compositions thereof include, but are not limited to, intradermal, intramuscular, intraperitoneal, intravenous, subcutaneous, intranasal, epidural, and oral routes. The antibody or antigen binding fragments thereof, or cell inhibiting agents, and therapeutic compositions thereof may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The compositions and antibodies of the invention may be administered together with a second moiety for example a therapeutic molecule. Administration may be concurrent or sequential. The second moiety may be a chemotherapy agent, biologic, cytokine, small molecule, CAR-T therapy or radiotherapy treatment.

Chemotherapy agents include alkylating agents, plant alkaloids, antimetabolites, anthracyclines, topoisomerase inhibitors and corticosteroids. For example, the chemotherapy can include vinorelbine, cisplatin, carboplatin, gemcitabine, paclitaxel, topotecan, docetaxel, irinotecan, pemetrexed, etoposide, or any combination thereof.

A biologic may be an antibody therapy, for example an antibody that targets a checkpoint inhibitor, such as PD-1 (e.g. Pembrolizumab, Nivolumab or Cemiplimab), PD-L1 (e.g. Atezolizumab, Avelumab or Durvalumab), PD-L2, LAG-3 (e.g. Relatlimab), Tim-3 or CTLA4 (e.g. Ipilimumab).

The small molecule therapy may be Pexidartinib.

In another embodiment, the second moity is a label, for example a fluorescent molecule, β-galactosidase, luciferase molecules, chemical dyes, fluorophores or a radioisotope.

In another aspect, the agents, antibodies or antigen-binding fragments thereof of the invention are modified to increase half-life, for example by a chemical modification, especially by PEGylation, or by incorporation in a liposome, or using a serum albumin protein or an antibody or antibody fragment that binds human serum albumin. Increased half-life can also be conferred by conjugating the molecule to an antibody fragment. The term “half-life” as used herein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms.

Half-life may be increased by at least 1.5 times, preferably at least 2 times, such as at least 5 times, for example at least 10 times or more than 20 times, greater than the half-life of the corresponding antibodies of the invention. For example, increased half-life may be more than 1 hours, preferably more than 2 hours, more preferably more than 6 hours, such as more than 12 hours, or even more than 24, 48 or 72 hours, compared to the antibody of the invention. The in vivo half-life of an amino acid sequence, compound or polypeptide of the invention can be determined in any manner known per se, such as by pharmacokinetic analysis. Suitable techniques will be clear to the person skilled in the art. Half-life can for example be expressed using parameters such as the t1/2-alpha t1/2-beta and the area under the curve (AUC).

Preferably, the dual targeting therapy described herein will provide a benefit to the treatment of a CD33+B7H4+ malignancy in a subject in need thereof. For example, the dual targeting therapy may have an additive or synergistic effect on the treatment of a malignancy in a subject in need thereof. A dual targeting therapy is defined as affording an “additive effect”, “synergistic effect” or a “synergistic treatment” if the effect is therapeutically superior, as measured by, for example, the extent of the response (e.g., apoptosis or cell viability), the response rate, the time to disease progression or the survival period, to that achievable on dosing one or other of the components of the dual targeting therapy at its conventional dose. For example, the effect of the dual targeting therapy is additive if the effect is therapeutically superior to the effect achievable with an antibody or antigen binding fragments thereof that specifically binds to CD33, or B7H4 alone. For example, the effect of the combination treatment may be synergistic if the effect of the combination treatment supersedes the effect of the individual treatments added together. Further, the effect of the combination is beneficial (e.g., additive or synergistic) if a beneficial effect is obtained in a group of subjects that does not respond (or responds poorly) to a cell-inhibiting agent that specifically binds to CD33 alone or a cell-inhibiting agent that specifically binds to B7H4 alone. In addition, the effect of the combination treatment is defined as affording a benefit (e.g. additive or synergistic effect) if one of the components is dosed at its conventional dose and the other component is dosed at a reduced dose and the therapeutic effect, as measured by, for example, the extent of the response, the response rate, the time to disease progression or the survival period, is equivalent to or better than that achievable on dosing conventional amounts of either one of the components of the combination treatment.

As used herein, “killing of a target cell” relates to an inhibition of protein synthesis, for example such that cell viability is reduced, or an induction of apoptosis resulting in elimination or death of target cells. Assays to determine cell killing and apoptosis are well known in the art. Cytotoxicity assays assess the number of live and dead cells in a population after treatment with a pharmacological substance (e.g., an LDH cytotoxicity assay, or a live-dead cell assay). Apoptosis assays assess how cells are dying by measuring markers that are activated upon cell death (e.g., a PS exposure assay, a caspase activation assay, a DNA fragmentation assay, a GSH/GSSG determination, a LDH cytotoxicity assay, a live-dead cell assay, or a non-caspase protease activation assay).

As used herein “inhibit the cell growth” (e.g., referring to target cells) refers to any measurable decrease in the growth or proliferation of a target cell when contacted with the antibody or antigen binding fragments thereof, or cell inhibiting agents, according to the present invention as compared to the growth of the same cell not in contact with the antibody or antigen binding fragments thereof, or cell inhibiting agents, according to the present disclosure, e.g., the inhibition of growth of a cell by at least about 10%, 20%, 30%, 40%, 50%, 60%, 70%, 80%, 90%, 99%, or 100%. Assays to determine cell viability or proliferation are well known in the art. Cell viability assays assess how healthy the cells are by measuring markers of cellular activity (e.g., an ATP and ADP determination assay, a cell cycle assay, a cell proliferation assay, a cell viability assay, an LHD cytotoxicity assay, or a live-dead cell assay). Cell proliferation assays assess the growth rate of a cell population or to detect daughter cells in a growing population (e.g., a cell cycle assay, a cell proliferation assay, a cell viability assay, or a senescence assay).

As used herein, “CD33 expressing cell” and “CD33+ cell” refers to a cell with CD33 as surface antigen. As used herein, “B7H4 expressing cell” and “B7H4+ cell” refers to a cell with B7H4 as surface antigen. As used herein, “CD33 and B7H4 expressing cell” and “CD33+B7H4+ cell” refers to a cell with both CD33 and B7H4 as surface antigens.

As used herein “target cell” refers to a cell or cell-type characterised by the expression or overexpression of the two target molecules CD33 and B7H4. Any type of cell expressing both CD33 and B7H4 may be envisaged as a target cell for treatment with the antibody or antigen binding fragments thereof, or cell inhibiting agents, of the invention. In certain embodiments, the cell is a tumour-associated immune cell, for example a tumour-associated immune cells associated with a malignancy, e.g. a cancer such as an ovarian, bladder or lung cancer, AML or another cancer as mentioned herein.

In certain embodiments, the antibody or antigen binding fragments thereof, or agents, e.g. cell inhibiting agents, described herein are capable of inducing CD33 receptor mediated internalisation of said antibody or antigen binding fragments thereof into a CD33+ cell, and/or B7H4 receptor mediated internalisation of said the antibody or antigen binding fragments thereof, or agents, e.g. cell inhibiting agents, into a B7H4+ cell. In certain embodiments, the antibody or antigen binding fragments thereof, or agents, e.g. cell inhibiting agents, is an antibody or antigen binding fragments thereof that specifically binds to both CD33 and B7H4 and is capable of inducing internalisation of the agent into a CD33+B7H4+ cell upon binding of both CD33 and B7H4 on a cell surface.

As used herein, “CD33 receptor mediated internalisation” refers to being taken up by (i.e., entry of) a CD33+ cell upon binding to CD33 on the cell surface. For therapeutic applications, internalisation in vivo is contemplated. As used herein, “B7H4 receptor mediated internalisation” refers to being taken up by (i.e., entry of) a B7H4+ cell upon binding to B7H4 on the cell surface.

For therapeutic applications, the concentration of the antibodies or antigen binding fragments or agents, e.g. cell inhibiting agents employed should be sufficient for the antibody or antigen binding fragments or cell inhibiting agents to be internalised and kill an CD33+B7H4+ cancer cell, such as cancer cell, e.g. an ovarian, bladder, lung cancer cell or an AML cell. Depending on the potency of the antibody or antigen binding fragments thereof, or cell inhibiting agents, in some instances, the uptake of a single molecule into the cell is sufficient to kill the target cell to which the agent binds.

In certain embodiments, the antibody or antigen binding fragments thereof, or cell inhibiting agents, of the invention may be antibody drug conjugates (ADCs), small-molecule drug conjugates (SMDCs), immunotoxins, peptide and non-peptide conjugates, imaging agents, therapeutic vaccines, nanoparticles.

The terms “antibody” or “antibodies” as used herein refer to molecules or active fragments of molecules that bind to known antigens, particularly to immunoglobulin molecules and to immunologically active portions of immunoglobulin molecules, i.e., molecules that contain a binding site that immunospecifically binds an antigen (i.e., CD33+ or B7H4). The immunoglobulin according to the invention can be of any class (IgG, IgM, IgD, IgE, IgA and IgY) or subclass (e.g., IgG1, IgG2, IgG3, IgG4, IgA1 and IgA2) or subclasses (isotypes) of immunoglobulin molecule (e.g., IgG in IgG1, IgG2, IgG3, and IgG4, or IgA in IgA1 and IgA2).

As antibodies can be modified in a number of ways, the term “antigen-binding protein” or “antibody” should be construed as covering antibody fragments, derivatives, functional equivalents and homologues of antibodies, including any polypeptide comprising an immunoglobulin binding domain such as the antibodies described herein. The terms also extends to different antibody formats, such as formats containing a Fab region and an scFV region as in specific aspects of the invention.

In a full-length antibody, each heavy chain is comprised of a heavy chain variable region or domain (abbreviated herein as HCVR, VH or V_{H}) and a heavy chain constant region. The heavy chain constant region is comprised of three domains, C_{H}1, C_{H}2 and C_{H}3. Each light chain is comprised of a light chain variable region or domain (abbreviated herein as LCVR, VL or V_{L}) and a light chain constant region. The light chain constant region is comprised of one domain, CL.

Antibodies may include the kappa (κ) and lambda (A) light chains and the alpha (IgA), gamma (IgG1, IgG2, IgG3, IgG4), delta (IgD), epsilon (IgE) and mu (IgM) heavy chains, or their equivalents in other species. Full-length immunoglobulin “light chains” (usually of about 25 kDa or usually about 214 amino acids long) consist of a variable region of approximately 110 amino acids at the NH2-terminus and a kappa or lambda constant region at the COOH-terminus. Full-length immunoglobulin “heavy chains” (usually of about 50 kDa or 446 amino acids long), likewise consist of a variable region (of about 116 amino acids) and one of the aforementioned heavy chain constant regions, e.g. gamma (of about 330 amino acids).

Light or heavy chain variable regions are generally composed of a “framework” region (FR) interrupted by three hypervariable regions, also called CDRs. The extent of the framework region and CDRs have been precisely defined. The sequences of the framework regions of different light and heavy chains are relatively conserved within a species. The framework region of an antibody, i.e. the combined framework regions of the constituent light and heavy chains, serves to position and align the CDRs. The CDRs are primarily responsible for binding to an epitope of an antigen. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

Different definitions of the CDRs are commonly in use. The method described by Kabat is the most commonly used and CDRs are based on sequence variability (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk, J. Mol. Biol. 196:901-917 (1987)). The Kabat numbering system is generally used when referring to a residue in the variable domain (approximately residues 1-107 of the light chain and residues 1-113 of the heavy chain). Another system is the ImMunoGeneTics (IMGT) numbering scheme (Lefranc et al., Dev. Comp. Immunol., 29, 185-203 (2005)). According to the IMGT numbering scheme, a CDR is a loop region of a variable domain, delimited according to the IMGT unique numbering for V domain. There are three CDR-IMGT in a variable domain: CDR1-IMGT (loop BC), CDR2-IMGT (loop C′C″), and CDR3-IMGT (loop FG).

Heavy chain CDRs are designated HCDR1, HCDR2 and HCDR3. Light chain CDRs are designated LCDR1, LCDR2 and LCDR3.

“Framework” or “FR” refers to variable domain residues other than hypervariable region (UVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

In one embodiment, the antibody is comprised of four polypeptide chains, two heavy (H) chains and two light (L) chains, or any functional fragment, mutant, variant, or derivation thereof, which retains the essential epitope binding features of an Ig molecule.

The term “antibody” is not only inclusive of antibodies generated by methods comprising immunisation, but also includes any polypeptide, e.g., a recombinantly expressed polypeptide, which is made to encompass at least one CDR capable of specifically binding to an epitope on an antigen of interest. Hence, the term applies to such molecules regardless whether they are produced in vitro, in cell culture, or in vivo. Methods of producing polyclonal and monoclonal antibodies are known in the art.

It is possible to take monoclonal and other antibodies and use techniques of recombinant DNA technology to produce other antibodies or chimeric molecules which generally retain the specificity of the original antibody. Such techniques may involve introducing the CDRs into a different immunoglobulin framework, or grafting variable regions onto a different immunoglobulin constant regions. Alternatively, a hybridoma or other cell producing an antibody molecule may be subject to genetic mutation or other changes, which may or may not alter the binding specificity of antibodies produced.

Within the scope of the present invention the terms “antibody” or “antibodies” include human and humanised antibodies as well as antigen binding fragments thereof. As used herein, antibody fragments are antigen binding fragments. Examples of antigen binding fragments of molecules that bind to known antigens include Fab, Fab′, F(ab′)2, F(ab′)3, Fabc, Fd, single chain Fv (scFv), (scFv) 2, Fv, scFv-Fc, heavy chain only antibody, diabody, tetrabody, triabody, minibody, or antibody mimetic protein s, including the products of a Fab immunoglobulin expression library and epitope-binding fragments of any of the antibodies and fragments mentioned above. Thus, the antibody fragment/antigen-binding fragment may comprise or consist of any of these fragments.

The “Fab fragment” of an antibody (also referred to as fragment antigen binding) contains the constant domain (CL) of the light chain and the first constant domain (CHI) of the heavy chain along with the variable domains VL and VH on the light and heavy chains respectively. The variable domains comprise the complementarity determining loops (CDR, also referred to as hypervariable region) that are involved in antigen binding. Fab′ fragments differ from Fab fragments by the addition of a few residues at the carboxy terminus of the heavy chain CHI domain including one or more cysteines from the antibody hinge region.

As used herein, the term “single-chain” refers to a molecule comprising amino acid monomers linearly linked by peptide bonds. In certain embodiments, one of the antigen binding moieties, e.g., antigen binding polypeptide construct, is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule. Fv fragments (˜25 kDa) consist of the two variable domains, VH and VL. Naturally, VH and VL domain are non-covalently associated via hydrophobic interaction and tend to dissociate. However, stable fragments can be engineered by linking the domains with a hydrophilic flexible linker to create a single chain Fv (scFv). In certain other embodiments, one of the antigen binding moieties is a single-chain Fv molecule (scFv).

“Single-chain Fv” or “scFv” antibody fragments comprise the VH and VL domains of an antibody, wherein these domains are present in a single polypeptide chain. In one embodiment, the Fv polypeptide further comprises a polypeptide linker between the VH and VL domains which enables the scFv to form the desired structure for antigen binding.

The term “Fc” or “Fc domain” or “Fc region” or “Fc construct” herein is used to define a C-terminal region of an immunoglobulin heavy chain. The term includes native sequence Fc regions and variant Fc regions.

“Fc region”, as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences.

The Fc sequence of an immunoglobulin generally comprises two constant domains, a CH2 domain and a CH3 domain, and optionally comprises a CH4 domain. By “Fc polypeptide” herein is meant one of the polypeptides that make up an Fc region. An Fc polypeptide may be obtained from any suitable immunoglobulin, such as IgG1, IgG2, IgG3, or IgG4 subtypes, IgA, IgE, IgD or IgM. In some embodiments, an Fc polypeptide comprises part or all of a wild type hinge sequence (generally at its N terminus). In some embodiments, an Fc polypeptide does not comprise a functional or wild type hinge sequence. The antibody may comprise a CH2 domain. The CH2 domain is for example located at the N-terminus of the CH3 domain, as in the case in a human IgG molecule. The CH2 domain of the antibody is in one embodiment the CH2 domain of human IgG1, IgG2, IgG3, or IgG4, e.g. the CH2 domain of human IgG1. The sequences of human IgG domains are known in the art.

As used herein the term “humanised antibody” or “humanised version of an antibody” refers to antibodies in which the framework or “complementarity determining regions” (CDR) have been modified to comprise the CDR of an immunoglobulin of different specificity as compared to that of the parent immunoglobulin. In some exemplary embodiments, the CDRs of the VH and VL are grafted into the framework region of human antibody to prepare the “humanised antibody.” See e.g., Riechmann, L., et al., Nature 332 (1988) 323-327; and Neuberger, M. S., et al., Nature 314 (1985) 268-270. The heavy and light chain variable framework regions can be derived from the same or different human antibody sequences. The human antibody sequences can be the sequences of naturally occurring human antibodies. Human heavy and light chain variable framework regions are listed e.g., in Lefranc, M.-P., Current Protocols in Immunology (2000)—Appendix 1P A. 1P.1-A. 1P.37 and are accessible via IMGT, the international ImMunoGeneTics information System® (http://imgt.cines.fr) or via http://vbase.mrc-cpe.cam.ac.uk, for example. Optionally the framework region can be modified by further mutations. Exemplary CDRs correspond to those representing sequences recognising the antigens noted above for chimeric antibodies. In some embodiments, such humanised version is chimerised with a human constant region. The term “humanised antibody” as used herein also comprises such antibodies which are modified in the constant region to generate the properties according to the disclosure, especially in regard to Clq binding and/or FcR binding, e.g., by “class switching” i.e., change or mutation of Fc parts (e.g., from IgG1 to IgG4 and/or IgG1/IgG4 mutation).

As used herein the term “human antibody” is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunisation, of producing a full repertoire or a selection of human antibodies in the absence of endogenous immunoglobulin production. Transfer of the human germ-line immunoglobulin gene array in such germ-line mutant mice results in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Brueggemann, M. D., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole, A., et al. and Boerner, P., et al. are also available for the preparation of human monoclonal antibodies (Cole, A., et al., Monoclonal Antibodies and Cancer Therapy, Liss, A. R. (1985) p. 77; and Boerner, P., et al., J. Immunol. 147 (1991) 86-95). As already mentioned, according to the instant disclosure the term “human antibody” as used herein also comprises such antibodies which are modified in the constant region to generate the properties according to the disclosure, for example in regard to Clq binding and/or FcR binding, e.g., by “class switching” i.e., change or mutation of Fc parts (e.g., from IgG1 to IgG4 and/or IgG1/IgG4 mutation).

As used herein the term “antibody fragment” refers to a portion of a full-length antibody, the term “antigen binding fragments” refers to a variable domain thereof, or at least an antigen binding site thereof, for example the CDRs. Examples of antibody fragments include diabodies, single-chain antibody molecules, and multispecific antibodies formed from antibody fragments. scFv antibodies are, e.g., described in Huston, J. S., Methods in Enzymol. 203 (1991) 46-88. Antibody fragments can be derived from an antibody of the present invention by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

As used herein the term “bispecific antibodies” refers to antibodies that bind to two (or more) different antigens. A bispecific antibody typically comprises at least two different variable domains, wherein each variable domain is capable of specifically binding to a separate antigen. In certain aspects, the bispecific antibodies of the invention are human antibodies. As used herein, the expression “bispecific antibody” means a protein, polypeptide or molecular complex comprising at least a first antigen-binding domain and a second antigen-binding domain. Each antigen-binding domain within the bispecific antibody comprises at least one CDR that alone, or in combination with one or more additional CDRs, specifically binds to a particular antigen. In the context of the present invention, the first antigen-binding domain specifically binds a first antigen (e.g., CD33), and the second antigen-binding domain specifically binds a second, distinct antigen (e.g., B7H4). In certain aspects, the bispecific molecules are capable of simultaneously binding to human CD33 and human B7H4.

In certain embodiments the bispecific antibodies may be referred to as “anti-CD33×B7H4” or “anti-CD33/anti-B7H4” and so forth. A bispecific antibody may have a sequence as shown in the examples. The CD33 binding portion may comprise Gemtuzumab or a fragment thereof. The CD33 binding portion may comprise SEQ ID. 3 and/or 4 or SEQ ID. 7 and/or 8.

A bispecific antibody may have a drug-to-antibody ratio (DAR) of 3 to 7, e.g. 3, 4, 5, 6, or 7.

Any bispecific antibody format or technology may be used to make the bispecific antibodies. Specific exemplary bispecific formats that can be used in the context of the present invention include, without limitation, e.g., scFv-based or diabody bispecific formats, IgG-scFv fusions, Fc-Fab-scFv fusions dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, Mab2 bispecific formats (see, e.g., Klein et al. 2012, imAbs 4:6, 1-1 1, and references cited therein, for a review of the foregoing formats) and Fab-based bispecific formats. In certain embodiments, the bispecific antibody is a Fab-based anti-CD33×B7H4 bispecific.

As used herein the term “specific” and “specifically” are used interchangeably to indicate that biomolecules other than CD33 or B7H4 (or where the biomolecule is a bispecific molecule both CD33 and B7H4) do not significantly bind to the antibody. In some embodiments, the level of binding to a biomolecule other than CD33 or B7H4 is negligible (e.g., not determinable) by means of ELISA or an affinity determination.

By “negligible binding” a binding is meant, which is at least about 85%, particularly at least about 90%, more particularly at least about 95%, even more particularly at least about 98%, but especially at least about 99% and up to 100% less than the binding to CD33, or B7H4.

The binding affinity of an antibody to a peptide or epitope may be determined with a standard binding assay, such as surface plasmon resonance technique (BIAcore®, GE-Healthcare Uppsala, Sweden). The term “surface plasmon resonance,” as used herein, refers to an optical phenomenon that allows for the analysis of real-time bispecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

In one embodiment, the antibody or antigen binding fragments thereof, or cell inhibiting agents, of the invention are capable of mediating antibody dependent cell cytotoxicity. Antibody dependent cellular cytotoxicity (ADCC) is an immune effector cell mediated mechanism which may contribute to anti-tumour activity of monoclonal antibodies (Weiner G J. Monoclonal antibody mechanisms of action in cancer. Immunol Res. 2007, 39 (1-3): 271-8). The relevance of ADCC for anti-tumour efficacy has been demonstrated in preclinical models, e.g., in mouse tumour models (e.g., Clynes R A, Towers T L, Presta L G, Ravetch J V. Inhibitory Fc receptors modulate in vivo cytotoxicity against tumour targets. Nat Med. 2000 April; 6 (4): 443-6). Data from clinical trials support the relevance of ADCC for clinical efficacy of therapeutic antibodies (e.g., Weng W K, Levy R Two immunoglobulin G fragment C receptor polymorphisms independently predict response to rituximab in patients with follicular lymphoma. J Clin Oncol. 2003 Nov. 1; 21 (21): 3940-7. Epub 2003 Sep. 15). Interactions of monoclonal antibodies with Fc receptors on immune cells contribute to ADCC. The Fc of antibodies can be modified in order to display enhanced affinity to Fc receptors (e.g., Presta L G Engineering of therapeutic antibodies to minimise immunogenicity and optimise function. Adv Drug Deliv Rev. 2006 Aug. 7; 58 (5-6): 640-56. Epub 2006 May 23). Such enhanced affinity to Fc receptors results in increased ADCC activity which may lead to increased anti-tumour efficacy in patients.

Thus, in one embodiment, the bispecific antibody comprises an Fc region. Thus, the cell inhibiting agent may be a bispecific antibody that binds CD33 and B7H4 and which comprises an Fc region.

In an alternative embodiment, the antigen binding fragments thereof of the invention are immunoresponsive cells which expresses a chimeric antigen T cell receptor protein (CAR), wherein the chimeric T cell receptor protein specifically binds to CD33 and B7H4. In one embodiment immunoresponsive cell is bispecific and which a chimeric antigen T cell receptor protein (CAR), wherein the chimeric T cell receptor protein specifically binds to CD33 and a chimeric antigen T cell receptor protein (CAR), wherein the chimeric T cell receptor protein specifically binds to B7H4.

In some embodiments, the immunoresponsive cell is autologous to the subject. In another embodiment, the immunoresponsive cell is not autologous to the subject. In a particular embodiment, the immunoresponsive cell is a T cell and is autologous to the subject to be treated.

In some embodiments, the antibody or antigen binding fragments thereof, or agents, e.g. cell inhibiting agents, comprises a binding portion (i.e. a CD33 binding portion and a B7H4 binding portion) and a payload, for example a cell killing agent, an immune-modulating payload or a light activatable payload. In certain embodiments, the cell binding portion is an antibody or antigen binding fragments thereof. In particular embodiments the cell binding portion is an antibody or antigen binding fragments thereof.

In some embodiments, the antibody or antigen binding fragments thereof, or agents, e.g. cell inhibiting agents, further comprises (or is incorporated or associated with) a cytotoxic or cytostatic agent, i.e., a compound that kills or inhibits tumour cells. Such agents may impart their cytotoxic and cytostatic effects by mechanisms including tubulin binding, DNA binding, proteasome and/or topoisomerase inhibition.

The cytotoxic or cytostatic agent may be, for example, a peptide toxin, a small molecule toxin or a radioisotope. This is also referred to herein as drug or cytotoxic payload.

As used herein, an “ADC” is an antibody drug conjugate.

In one embodiment the cytotoxic or cytostatic agent may be a tubulin inhibitor; or a DNA interacting agent. Tubulin inhibitors modulate tubulin polymerisation. DNA interacting agents target cellular DNA.

In an embodiment the cytotoxic or cytostatic agent is a tubulin inhibitor. In an embodiment, the tubulin inhibitor is selected from the group consisting of: (a) an auristatin; and (b) a maytansine derivative. In an embodiment, the cytotoxic or cytostatic agent is an auristatin. Auristatins include synthetic derivatives of the naturally occurring compound Dolastatin-10. Auristatins are a family of antineoplastic/cytostatic pseudopeptides. Dolastatins are structurally unique due to the incorporation of 4 unusual amino acids (Dolavaine, Dolaisoleuine, Dolaproine and Dolaphenine) identified in the natural biosynthetic product. In addition, this class of natural product has numerous asymmetric centres defined by total synthesis studies by Pettit et al (U.S. Pat. No. 4,978,744). It would appear from structure activity relationships that the Dolaisoleuine and Dolaproine residues appear necessary for antineoplastic activity (U.S. Pat. Nos. 5,635,483 and 5,780,588). In an embodiment, the auristatin is selected from the group consisting of: Auristatin E (AE); Monomethylauristatin E (MMAE); Auristatin F (MMAF); vcMMAE; vcMMAF; mcMMAE and mcMMAF. In an embodiment, the cytotoxic or cytostatic agent is a maytansine or a structural analogue of maytansine. In an embodiment, the cytotoxic or cytostatic agent is a maytansine. Maytansines include structurally complex antimitotic polypetides. Maytansines are potent inhibitors of microtubulin assembly which leads towards apoptosis of tumour cells. In an embodiment the maytansine is selected from the group consisting of: Mertansine (DM1); and a structural analogue of maytansine such as DM3 or DM4. Preferably, the drug is MMAE, MMAF or auristatin MMAF.

In an embodiment, the cytotoxic or cytostatic agent is DNA interacting agent. In an embodiment, the DNA interacting agent is selected from the group consisting of: (a) calicheamicins, (b) duocarmycins and (c) pyrrolobenzodiazepines (PBDs). In an embodiment, the cytotoxic or cytostatic agent is a calicheamicin. Calicheamicin is a potent cytotoxic agent that causes double-strand DNA breaks, resulting in cell death. Calicheamicin is a naturally occurring enediyne antibiotic (A. L. Smith et al, J. Med. Chem., 1996, 39, 11, 2103-2117). Calicheamicin was found in the soil microorganism Micromonosporaechinospora. In an embodiment, the calicheamicin is calicheamicin gamma 1. In an embodiment, the drug is a duocarmycin. Duocarmycins are potent anti-tumour antibiotics that exert their biological effects through binding sequence-selectively in the minor groove of DNA duplex and alkylating the N3 of adenine (D. Boger, Pure & Appl. Chem., 1994, 66, 4, 837-844). In an embodiment, the duocarmycin is selected from the group consisting of: Duocarmycin A; Duocarmycin B1; Duocarmycin B2; Duocarmycin C1; Duocarmycin C2; Duocarmycin D; Duocarmycin S A; Cyclopropylbenzoindole (CBI) duocarmycin; Centanamycin; Rachelmycin (CC-1065); Adozelesin; Bizelesin; and Carzelesin. In an embodiment, the cytotoxic or cytostatic agent is a pyrrolobenzodiazepine. Pyrrolobenzodiazepines (PBDs) are a class of naturally occurring anti-tumour antibiotics. Pyrrolobenzodiazepines are found in Streptomyces. PBDs exert their anti-tumour activity by covalently binding to the DNA in the minor groove specifically at purine-guanine-purine units. They insert on to the N2 of guanine via an aminal linkage and, due to their shape, they cause minimal disruption to the DNA helix. It is believed that the formation of the DNA-PBD adduct inhibits nucleic acid synthesis and causes excision-dependent single and double stranded breaks in the DNA helix. As synthetic derivatives the joining of two PBD units together via a flexible polymethylene tether allows the PBD dimers to cross-link opposing DNA strands producing highly lethal lesions. In an embodiment, the cytotoxic or cytostatic agent is a synthetic derivative of two pyrrolobenzodiazepines units joined together via a flexible polymethylene tether. In an embodiment, the pyrrolobenzodiazepine is selected from the group consisting of: Anthramycin (and dimers thereof); Mazethramycin (and dimers thereof); Tomaymycin (and dimers thereof); Prothracarcin (and dimers thereof); Chicamycin (and dimers thereof); Neothramycin A (and dimers thereof); Neothramycin B (and dimers thereof); DC-81 (and dimers thereof); Sibiromycin (and dimers thereof); Porothramycin A (and dimers thereof); Porothramycin B (and dimers thereof); Sibanomycin (and dimers thereof); Abbeymycin (and dimers thereof); SG2000; and SG2285.

In an embodiment, the cytotoxic or cytostatic agent is a drug that targets DNA interstrand crosslinks through alkylation. A drug that targets DNA interstrand crosslinks through alkylation is selected from: a DNA targeted mustard; a guanine-specific alkylating agent; and a adenine-specific alkylating agent. In an embodiment, the cytotoxic or cytostatic agent is a DNA targeted mustard. For example, the DNA targeted mustard may be selected from the group consisting of: an oligopyrrole; an oligoimidazole; a Bis-(benzimidazole) carrier; a Polybenzamide Carrier; and a 9-Anilinoacridine-4-carboxamide carrier.

In an embodiment, the cytotoxic or cytostatic agent is selected from the group consisting of: Netropsin; Distamycin; Lexitropsin; Tallimustine; Dibromotallimustine; PNU 157977; and MEN 10710.

In an embodiment, the cytotoxic or cytostatic agent is a Bis-(benzimidazole) carrier. Preferably, the drug is Hoechst 33258.

A guanine-specific alkylating agent is a highly regiospecific alkylating agents that reacts at specific nucleoside positions. In an embodiment, the cytotoxic or cytostatic agent is a guanine-specific alkylating agent selected from the group consisting of: a G-N2 alkylators; a A-N3 alkylator; a mitomycin; a carmethizole analogue; a ecteinascidin analogue. In an embodiment, the mitomycin is selected from: Mitomycin A; Mitomycin C; Porfiromycin; and KW-2149. In an embodiment, the a carmethizole analogue is selected from: Bis-(Hydroxymethyl) pyrrolizidine; and NSC 602668. In an embodiment, the ecteinascidin analogue is Ecteinascidin 743.

Adenine-specific alkylating agents are regiospecific and sequence-specific minor groove alkylators reacting at the N3 of adenines in polypyrimidines sequences.

Cyclopropaindolones and duocamycins may be defined as adenine-specific alkylators. In an embodiment, the cytotoxic or cytostatic agent is a cyclopropaindolone analogue. Preferably, the drug is selected from: adozelesin; and carzelesin.

In an embodiment, the cytotoxic or cytostatic agent is a benz [e]indolone. Preferably, the cytotoxic or cytostatic agent is selected from: CBI-TMI; and iso-CBI.

In an embodiment, the cytotoxic or cytostatic agent is bizelesin. In an embodiment, the cytotoxic or cytostatic agent is a Marine Antitumour Drug. Marine Antitumour Drugs has been a developing field in the antitumour drug development arena (I. Bhatnagar et al, Mar. Drugs 2010, 8, P2702-2720 and T. L. Simmons et al, Mol. Cancer Ther. 2005, 4 (2), P333-342). Marine organisms including sponges, sponge-microbe symbiotic association, gorgonian, actinomycetes, and soft coral have been widely explored for potential anticancer agents.

In an embodiment, the cytotoxic or cytostatic agent is selected from: Cytarabine, Ara-C; Trabectedin (ET-743); and EribulinMesylate. In an embodiment, the EribulinMesylate is selected from: (E7389); Soblidotin (TZT 1027); Squalamine lactate; CemadotinPlinabulin (NPI-2358); Plitidepsin; Elisidepsin; Zalypsis; Tasidotin, Synthadotin; (ILX-651); Discodermolide; HT1286; LAF389; Kahalalide F; KRN7000; Bryostatin 1; Hemiasterlin (E7974); Marizomib; Salinosporamide A; NPI-0052); LY355703; CRYPTO 52; Depsipeptide (NSC630176); Ecteinascidin 743; Synthadotin; Kahalalide F; Squalamine; Dehydrodidemnin B; Didemnin B; Cemadotin; Soblidotin; E7389; NVP-LAQ824; Discodermolide; HTI-286; LAF-389; KRN-7000 (Agelasphin derivative); Curacin A; DMMC; Salinosporamide A; Laulimalide; Vitilevuamide; Diazonamide; Eleutherobin; Sarcodictyin; Peloruside A; Salicylihalimides A and B; Thiocoraline; Ascididemin; Variolins; Lamellarin D; Dictyodendrins; ES-285 (Spisulosine); and Halichondrin B.

The following cytotoxic or cytostatic agent are also encompassed by the present invention: Amatoxins (α-amanitin)-bicyclic octapeptides produced by basidiomycetes of the genus Amanita, e.g., the Green Deathcap mushroom; Tubulysins; Cytolysins; dolabellanins; Epothilone A, B, C, D, E, F. Epothilones-constitute a class of non-taxane tubulin polymerisation agents and are obtained by natural fermentation of the myxobacterium Sorangiumcellulosum. These moieties possess potent cytotoxic activity which is linked to the stabilisation of microtubules and results in mitotic arrest at the G2/M transition. Epothilones have demonstrated potent cytotoxicity across a panel of cancer cell lines and has often exhibited greater potency than paclitaxel (X.: Pivot et al, European Oncology, 2008; 4 (2), P42-45). In an embodiment, the drug is amatoxin. In an embodiment, the drug is tubulysin. In an embodiment, the drug is cytolysin. In an embodiment, the drug is dolabellanin. In an embodiment, the drug is epothilone.

The following cytotoxic or cytostatic agent are also encompassed by the present invention. In an embodiment, the drug is selected from: Doxorubicin; Epirubicin; Esorubicin; Detorubicin; Morpholino-doxorubicin; Methotrexate; Methopterin; Bleomycin; Dichloromethotrexate; 5-Fluorouracil; Cytosine-β-D-arabinofuranoside; Taxol; Anguidine; Melphalan; Vinblastine; Phomopsin A; Ribosome-inactivating proteins (RIPs); Daunorubicin; Vinca alkaloids; Idarubicin; Melphalan; Cis-platin; Ricin; Saporin; Anthracyclines; Indolino-benzodiazepines; 6-Mercaptopurine; Actinomycin; Leurosine; Leurosideine; Carminomycin; Aminopterin; Tallysomycin; Podophyllotoxin; Etoposide; Hairpin polyamides; Etoposide phosphate; Vinblastine; Vincristine; Vindesine; Taxotere retinoic acid; N8-acetyl spermidine; Camptothecin; Esperamicin; and Ene-diynes.

In one embodiment, the cell killing agent/portion is a peptide toxin, for example an auristatin such as Auristatin E (AE); Monomethylauristatin E (MMAE); Auristatin F (MMAF), vcMMAE, vcMMAF, mcMMAE and mcMMAF.

In another embodiment, the antibody or antigen binding fragments thereof, or cell inhibiting agents, comprises a binding portion and a payload, for example a cell killing agent, an immune-modulating payload or a light activatable payload, wherein the binding portion is an anti-CD33 anti-B7H4 bispecific antibody or binding portion thereof and a cell killing portion. In another embodiment, the antibody or antigen binding fragments thereof, or cell inhibiting agents, comprises a binding portion and a payload, for example a cell killing agent, an immune-modulating payload or a light activatable payload, wherein the binding portion is an anti-CD33 anti-B7H4 bispecific antibody or binding portion thereof and a payload, for example a cell killing agent, an immune-modulating payload or a light activatable payload.

In certain embodiments, the antibody or antigen binding fragments thereof, or cell inhibiting agents, comprises a binding portion that is conjugated to a payload, for example a cell killing agent, an immune-modulating payload or a light activatable payload. Such conjugates may be prepared by in vitro methods known to one of ordinary skill in the art. Techniques for conjugating cytotoxic or cytostatic agent to proteins, and in particular to antibodies, are well-known. (See, e.g., Alley et ah, Current Opinion in Chemical Biology 2010 14:1-9; Senter, Cancer J., 2008, 14 (3): 154-169.)

In certain embodiments, a linking group is used to conjugate the binding portion and the payload, for example a cell killing agent, an immune-modulating payload or a light activatable payload.

The linker can be cleavable under intracellular conditions, such that cleavage of the linker releases the payload, for example a cell killing portion, an immune-modulating payload or a light activatable payload from the binding portion in the intracellular environment. The cleavable linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in NTB-A-expressing cells. For example, a peptidyl linker that is cleavable by the thiol-dependent protease cathepsin-B, which is highly expressed in cancerous tissue, can be used {e.g., a linker comprising a Phe-Leu or a Val-Cit peptide).

The cleavable linker can be pH-sensitive, i.e., sensitive to hydrolysis at certain pH values.

Typically, the pH-sensitive linker is hydrolysable under acidic conditions. For example, an acid-labile linker that is hydrolysable in the lysosome (e.g., a hydrazone, semicarbazone, thiosemicarbazone, cis-aconitic amide, orthoester, acetal, ketal, or the like) can be used.

Other linkers are cleavable under reducing conditions (e.g., a disulfide linker). The cleavable linker can also be a malonate linker (Johnson et al, Anticancer Res. 15:1387-93, 1995), a maleimidobenzoyl linker (Lau et al, Bioorg-Med-Chem. 3:1299-1304, 1995), or a 3′-N-amide analogue (Lau et al, Bioorg-Med-Chem. 3:1305-12, 1995).

In some embodiments the linker can be a protease cleavable linker, for example a valine-citrulline, which may be cleaved by cathepsin B in the lysosome.

The linker also can be a non-cleavable linker, such as a maleimidoca-proyl (mc) linker or maleimido-alkylene- or maleimide-aryl linker that is directly attached to the therapeutic agent and released by proteolytic degradation of the binding portion.

The terms “conjugation” and “conjugate (d)” refer to chemical linkages, either covalent or non-covalent, which proximally associates one molecule of interest with a second molecule of interest.

The conjugate may be prepared by several routes, employing organic chemistry reactions, conditions, and reagents known to those skilled in the art, including: (1) reaction of a nucleophilic group or an electrophilic group of an antibody with a bivalent linker reagent, to form antibody-linker intermediate Ab-L, via a covalent bond, followed by reaction with an activated drug moiety D; and (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) may be employed with a variety of antibodies, drug moieties, and linkers to prepare the antibody-drug conjugates described here.

Several specific examples of methods of preparing bispecific antibodies and ADCs are known in the art. Traditional methods such as the hybrid hybridoma and chemical conjugation methods can be used in the preparation of the bispecific antibodies of the invention. Co-expression in a host cell of two antibodies, consisting of different heavy and light chains, leads to a mixture of possible antibody products in addition to the desired bispecific antibody, which can then be isolated by, e.g., affinity chromatography or similar methods.

Strategies favoring the formation of a functional bispecific, product, upon co-expression of different antibody constructs can also be used. Strategies for promoting heterodimerization are known in the art. One strategy to promote formation of heterodimers over homodimers is a “knob-into-hole” strategy in which a protuberance is introduced on a first heavy-chain polypeptide and a corresponding cavity in a second heavy-chain polypeptide, such that the protuberance can be positioned in the cavity at the interface of these two heavy chains so as to promote heterodimer formation and hinder homodimer formation.

Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent.

Additional nucleophilic groups can be introduced into antibodies through the reaction of lysines with 2-iminothiolane (Traut's reagent) resulting in conversion of an amine into a thiol.

Antibody-drug conjugates may also be produced by modification of the antibody to introduce electrophilic moieties, which can react with nucleophilic substituents on the linker reagent or drug. The sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the protein that can react with appropriate groups on the drug. In another embodiment, proteins containing N-terminal serine or threonine residues can react with sodium meta-periodate, resulting in production of an aldehyde in place of the first amino acid. Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

In another embodiment, the second moiety is a label, for example a fluorescent molecule, β-galactosidase, luciferase molecules, chemical dyes, fluorophores or a radioisotope.

There are several methods by which to produce recombinant antibodies which are known in the art. One of these is production in an E. coli expression system. In this embodiment, nucleic acids encoding the antibody or antigen-binding fragment thereof as described in previous aspects of the invention may be inserted into a plasmid and expressed in a suitable expression system. For example, the present invention includes methods for expressing an antibody or antigen-binding fragment thereof or immunoglobulin chain thereof in a host cell (e.g., bacterial host cell such as E. coli, CHO, HEK or other host cell according to the above described aspects of the invention).

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise. Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The invention also relates to the following non-limiting aspects.

1. A composition for use in the treatment of a malignancy, wherein the composition comprises a cell inhibiting agent that binds to CD33 and B7H4.

2. The composition for use according to aspect 1, wherein the composition is a non-immune suppressing treatment.

3. The composition for use according to aspect 2, wherein the non-immune suppressing treatment is non-myelosuppressing treatment.

4. The composition for use according to any preceding aspect, wherein the composition is an antibody or antigen binding fragment thereof.

5. The composition for use according to aspect 4, wherein the composition further comprises a cell killing agent.

6. The composition for use according to aspect 5, wherein the cell killing agent comprises a cytotoxin.

7. The composition for use according to aspect 6, wherein said cytotoxin is selected from:
i) a peptide toxin; or
ii) a chemical toxin.

8. The composition for use according to aspects 5 to 7, wherein the composition further comprises a linker for linking the cell killing agent to the cell inhibiting agent that binds to CD33 and B7H4 expressed on the cell surface.

9. The composition for use according to aspects 1 to 8, wherein the composition is a bispecific antibody drug conjugate.

10. The composition for use according to aspects 1 to 9, wherein the malignancy comprises a malignant disease associated with an increase in myeloid derived suppressor cells.

11. The composition for use according to aspects 1 to 10, wherein the malignancy is an ovarian cancer, an ovarian cancer derived cancer, AML or AML derived cancer.

12. The composition for use according to aspects 1 to 10, wherein the malignancy is selected from one of the following cancers: haematological cancers including Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukaemia (CLL), Myelodysplastic Syndrome (MDS), and Diffuse Large B cell Lymphoma (DLBCL); or Hepatocellular Carcinoma; or Gynaecological Cancers including Ovarian Cancer, Cervical Cancer, and Endometrial Cancer; lung cancer, bladder cancer or Renal Cell Carcinoma.

13 A combination of cell inhibiting agents for use in the treatment of a malignancy, wherein the cell inhibiting agents bind to CD33 and B7H4.

14. The combination for use according to aspect 13, wherein the combination is non-immune suppressing.

15. The combination for use according to aspect 14, wherein the non-immune suppressing treatment is non-myelosuppressing.

16. The combination for use according to any of aspects 13 to 15, wherein the cell inhibiting agents comprise antibodies or antigen binding fragment thereof.

17. The combination for use according to aspect 16, wherein the cell inhibiting agents further comprises a cell killing agent.

18. The combination for use according to aspect 17, wherein the cell killing agent comprises a cytotoxin.

19. The combination for use according to aspect 18, wherein said cytotoxin is selected from:
i) a peptide toxin; or
ii) a chemical toxin.

20. The combination for use according to aspects 17 to 19, wherein the cell inhibiting agents further comprises a linker for linking the cell killing agent to the cell inhibiting agent that binds to CD33 and B7H4 expressed on the cell surface.

21. The combination for use according to aspects 13 to 20, wherein the cell inhibiting agent is a antibody drug conjugate.

22. The combination for use according to aspects 13 to 21, wherein the malignancy comprises a malignant disease associated with an increase in myeloid derived suppressor cells.

23. The combination for use according to aspects 13 to 21, wherein the malignancy is an ovarian cancer, ovarian cancer derived cancer, AML or AML derived cancer.

24. The combination for use according to aspects 13 to 22, wherein the malignancy is selected from one of the following cancers: haematological cancers including Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukaemia (CLL), Myelodysplastic Syndrome (MDS) and Diffuse Large B cell Lymphoma (DLBCL); or Hepatocellular Carcinoma; or Gynaecological Cancers including Ovarian Cancer, Cervical Cancer and Endometrial Cancer; lung cancer, bladder cancer or Renal Cell Carcinoma.

25. A cell inhibiting agent for use in the treatment of a malignancy, wherein the cell inhibiting agent bispecifically binds to CD33 and B7H4.

26. The cell inhibiting agent for use according to aspect 25, wherein the treatment of the malignancy is non-immune suppressing.

27. The cell inhibiting agent for use according to aspect 26, wherein the non-immune suppressing treatment is non-myelosuppressing.

28. The cell inhibiting agent for use according to any one of aspects 25 to 27, wherein the cell inhibiting agent is an antibody or antigen binding fragment thereof.

29. The cell inhibiting agent for use according to aspect 28, wherein the cell inhibiting agent further comprises a cell killing agent.

30. The cell inhibiting agent for use according to aspect 29, wherein the cell killing agent comprises a cytotoxin.

31. The cell inhibiting agent for use according to aspect 30, wherein said cytotoxin is selected from:
i) a peptide toxin; or
ii) a chemical toxin.

32. The cell inhibiting agent for use according to aspects 29 to 31, wherein the cell inhibiting agent is a bispecific antibody drug conjugate.

33. The cell inhibiting agent for use according to aspects 25 to 32, wherein the malignancy is a malignant disease associated with an increase in myeloid derived suppressor cells.

34. The cell inhibiting agent for use according to aspect 33, wherein the malignancy is an ovarian cancer, an ovarian cancer derived cancer, an AML or AML derived cancer.

35. The cell inhibiting agent for use according to aspects 25 to 33, wherein the malignancy is selected from one of the following cancers: haematological cancers including Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukemia (CLL), Myelodysplastic Syndrome (MDS) and Diffuse Large B cell Lymphoma (DLBCL); or Hepatocellular Carcinoma; or Gynaecological Cancers including Ovarian Cancer, lung cancer, bladder cancer Cervical Cancer, and Endometrial Cancer; or Renal Cell Carcinoma.

### DETAILED DESCRIPTION OF THE FIGURES

Embodiments of the invention are described below, by way of example only with reference to and as illustrated in the following figures:

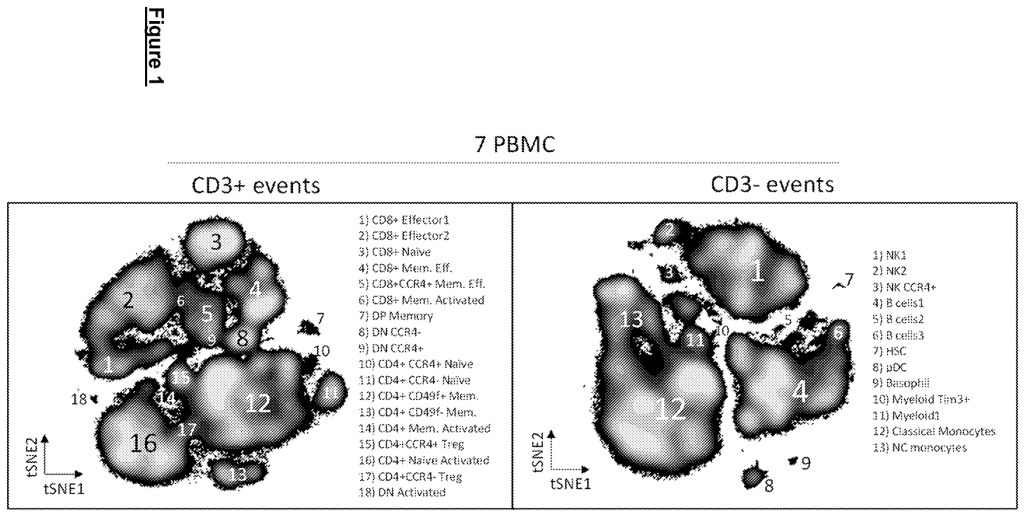

FIG. 1 is two bivariate tSNE plots of tSNE1 vs tSNE2 for each sample visualising cell density (lighter colours indicate higher cell density). The nomenclature of each PBMC cell type is marked on both plots and was defined by FlowSOM metaclusters from seven healthy patient samples of PBMC cells for both CD3+ and CD3− cell types. tSNE1 is shown on the x-axis and tSNE2 is shown on the y-axis. Each metacluster was identified by FlowSOM. The approximate location of each FlowSOM-identified cell type is numbered on the tSNE plots and corresponding cell-type nomenclature (according to its antigen expression) is listed.

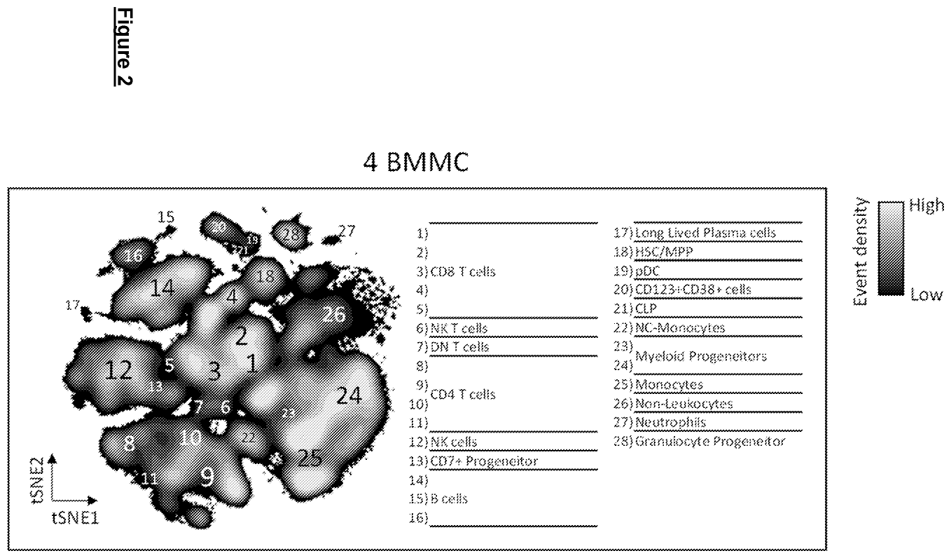

FIG. 2 is a bivariate tSNE plot of tSNE1 vs tSNE2 for each sample visualising cell density (lighter colours indicate higher cell density). The nomenclature of each BMMC cell type is marked on the plot and was defined by FlowSOM metaclusters from four healthy patient samples of BMMC cells. tSNE1 is shown on the x-axis and tSNE2 is shown on the y-axis. Each metacluster was identified by FlowSOM. The approximate location of each FlowSOM-identified cell type is numbered on the tSNE plot and corresponding cell-type nomenclature (according to its antigen expression) is listed.

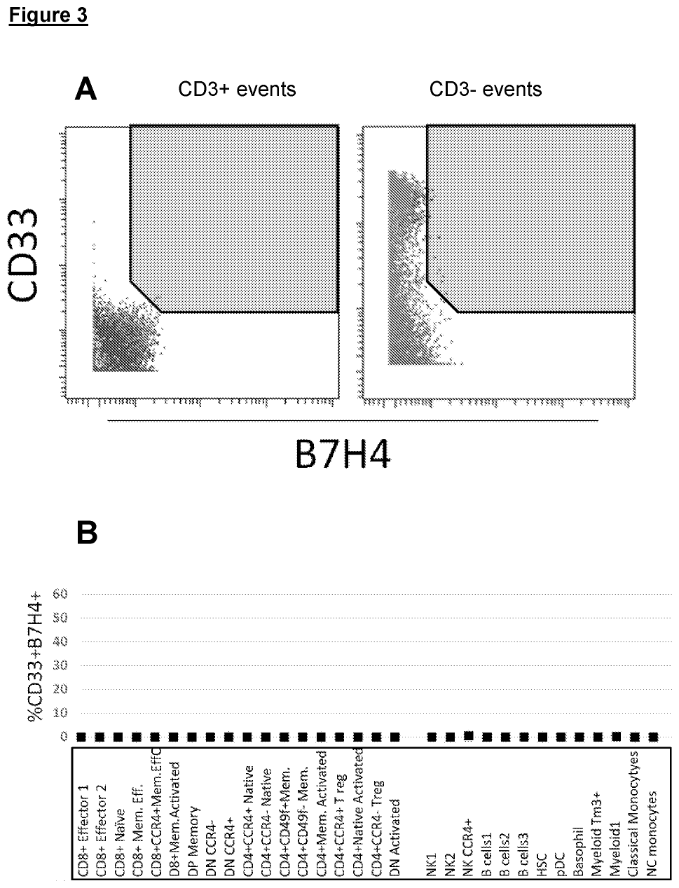

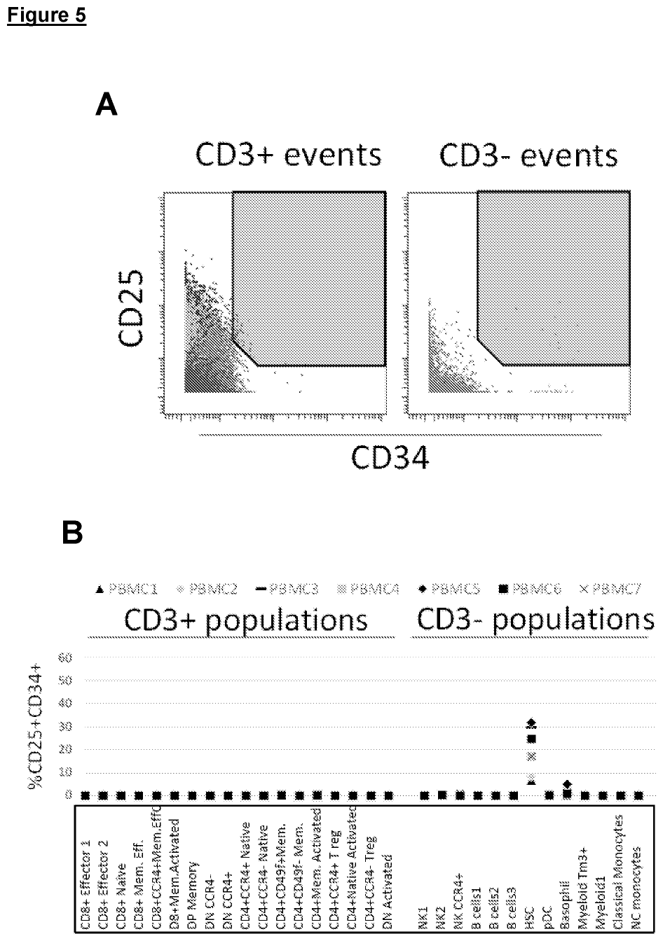

FIG. 3A are two bivariate plots (CD3+ and CD3− cell populations) which show each detection event corresponding to a single PBMC cell. The expression of one of either CD33 or B7H4 is shown on the y- and x-axis respectively. The plot shows the manual gating for dual positive events (grey shaded area). The gate threshold for non-specific antibody binding was calculated using the known single antigen positive cell types present. FIG. 3B is a graph that shows the percentage of cell detection events falling within the dual positive gate of FIG. 5A for each PBMC cell population identified by FlowSOM for each PBMC sample.

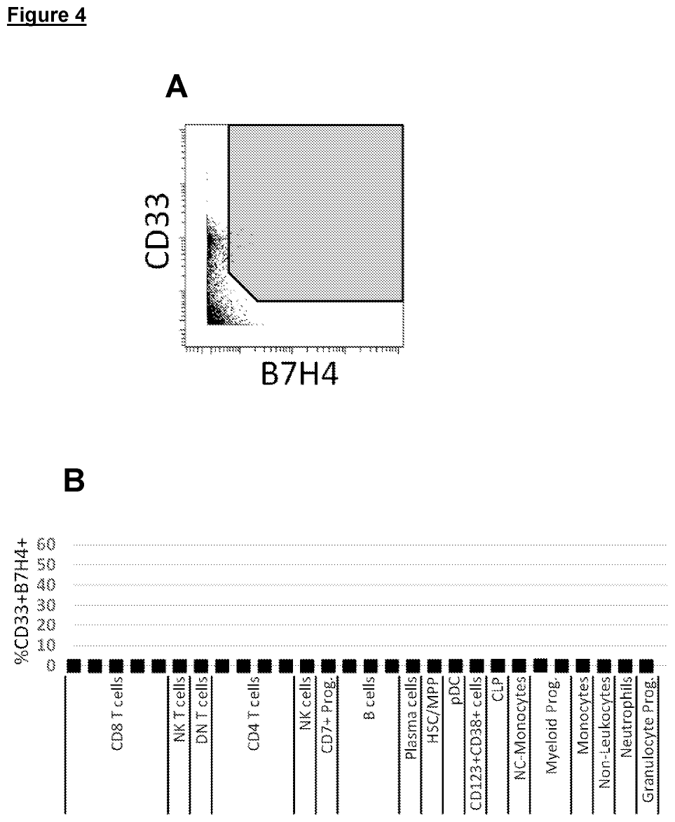

FIG. 4A is a bivariate plot which shows each detection event corresponding to a single BMMC cell. The expression of one of either CD33 or B7H4 is shown on the y- and x-axis respectively. The plot shows the manual gating for dual positive events (grey shaded area). The gate threshold for non-specific antibody binding was calculated using the known single antigen positive cell types present. FIG. 4B is a graph that shows the percentage of cell detection events falling within the dual positive gate of FIG. 4A for each BMMC cell population identified by FlowSOM for each BMMC sample.

FIG. 5A are two bivariate plots (CD3+ and CD3− cell populations) which show each detection event corresponding to a single PBMC cell. The expression of one of either CD25 or CD34 is shown on the y- and x-axis respectively. The plot shows the manual gating for dual positive events (grey shaded area). The gate threshold for non-specific antibody binding was calculated using the known single antigen positive cell types present. FIG. 5B is a graph that shows the percentage of cell detection events falling within the dual positive gate of FIG. 5A for each PBMC cell population identified by FlowSOM for each PBMC sample.

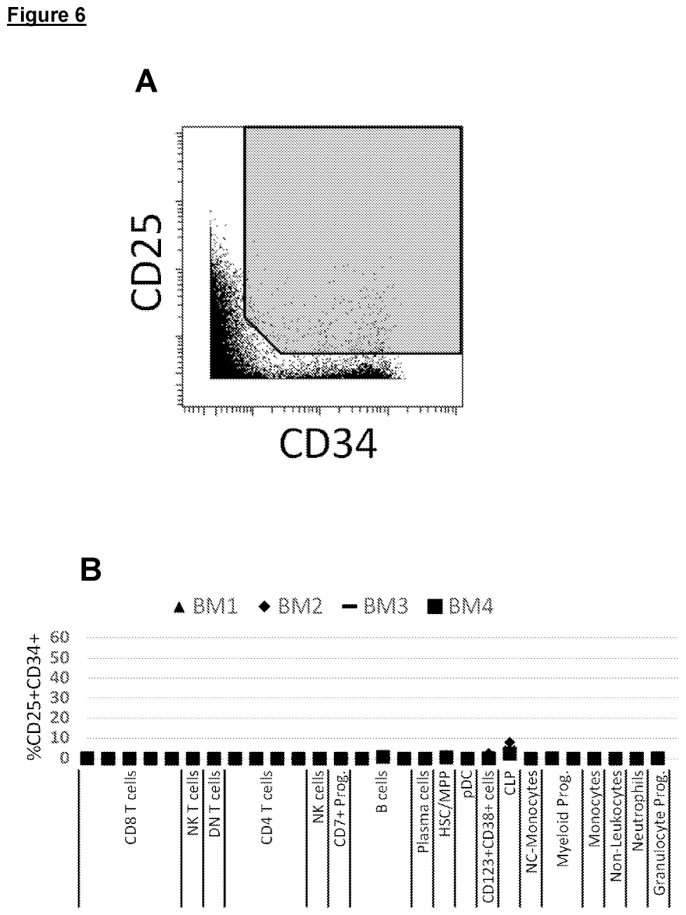

FIG. 6A is a bivariate plot which shows each detection event corresponding to a single BMMC cell. The expression of one of either CD25 or CD34 is shown on the y- and x-axis respectively. The plot shows the manual gating for dual positive events (grey shaded area). The gate threshold for non-specific antibody binding was calculated using the known single antigen positive cell types present. FIG. 6B is a graph that shows the percentage of cell detection events falling within the dual positive gate of FIG. 6A for each BMMC cell population identified by FlowSOM for each BMMC sample.

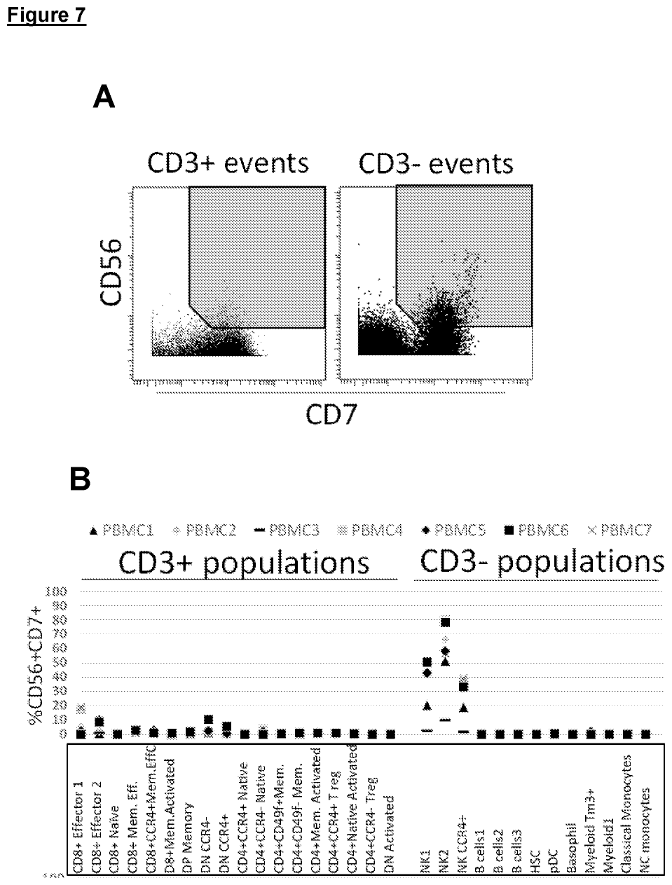

FIG. 7A are two bivariate plots (CD3+ and CD3− cell populations) which show each detection event corresponding to a single PBMC cell. The expression of one of either CD56 or CD7 is shown on the y- and x-axis respectively. The plot shows the manual gating for dual positive events (grey shaded area). The gate threshold for non-specific antibody binding was calculated using the known single antigen positive cell types present. FIG. 7B is a graph that shows the percentage of cell detection events falling within the dual positive gate of FIG. 7A for each PBMC cell population identified by FlowSOM for each PBMC sample.

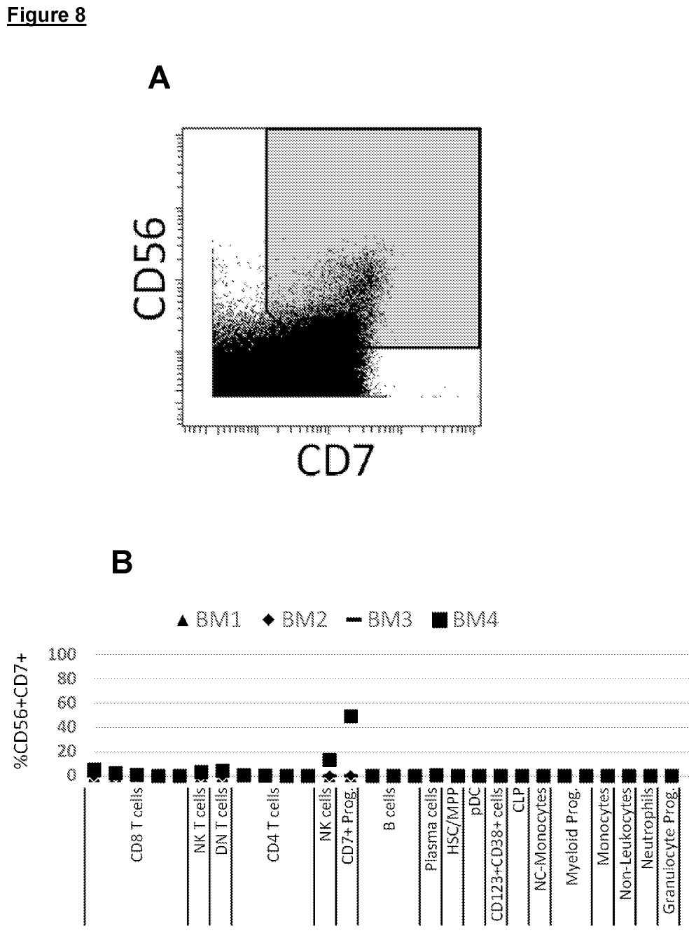

FIG. 8A is a bivariate plot which shows each detection event corresponding to a single BMMC cell. The expression of one of either CD56 or CD7 is shown on the y- and x-axis respectively. The plot shows the manual gating for dual positive events (grey shaded area). The gate threshold for non-specific antibody binding was calculated using the known single antigen positive cell types present. FIG. 8B is a graph that shows the percentage of cell detection events falling within the dual positive gate of FIG. 8A for each BMMC cell population identified by FlowSOM for each BMMC sample.

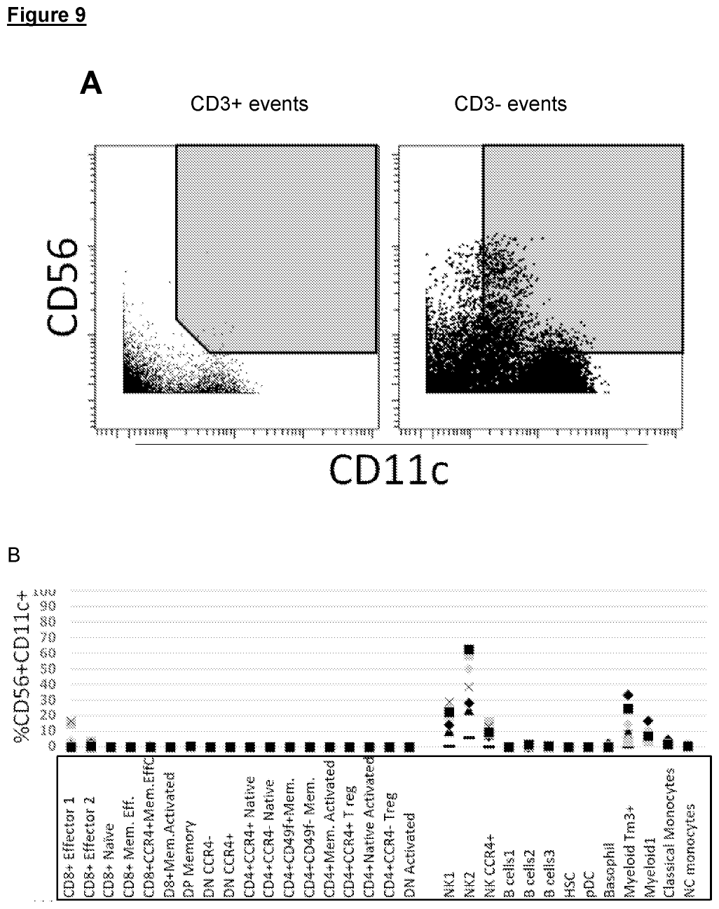

FIG. 9A are two bivariate plots (CD3+ and CD3− cell populations) which show each detection event corresponding to a single PBMC cell. The expression of one of either CD56 or CD11c is shown on the y- and x-axis respectively. The plot shows the manual gating for dual positive events (grey shaded area). The gate threshold for non-specific antibody binding was calculated using the known single antigen positive cell types present. FIG. 9B is a graph that shows the percentage of cell detection events falling within the dual positive gate of FIG. 9A for each PBMC cell population identified by FlowSOM for each PBMC sample.

FIG. 10A is a bivariate plot which shows each detection event corresponding to a single BMMC cell. The expression of one of either CD56 or CD11c is shown on the y- and x-axis respectively. The plot shows the manual gating for dual positive events (grey shaded area). The gate threshold for non-specific antibody binding was calculated using the known single antigen positive cell types present. FIG. 10B is a graph that shows the percentage of cell detection events falling within the dual positive gate of FIG. 10A for each BMMC cell population identified by FlowSOM for each BMMC sample.

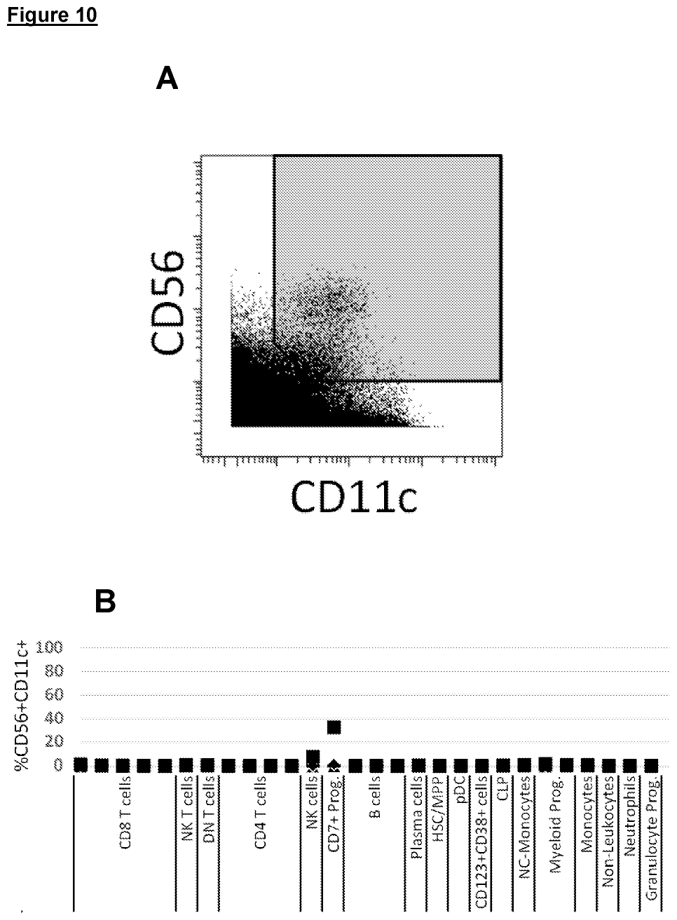

FIG. 11A are two bivariate plots (CD3+ and CD3− cell populations) which show each detection event corresponding to a single PBMC cell. The expression of one of either CD33 or CD371 is shown on the y- and x-axis respectively. The plot shows the manual gating for dual positive events (grey shaded area). The gate threshold for non-specific antibody binding was calculated using the known single antigen positive cell types present. FIG. 11B is a graph that shows the percentage of cell detection events falling within the dual positive gate of FIG. 11A for each PBMC cell population identified by FlowSOM for each PBMC sample.

FIG. 12A is a bivariate plot which shows each detection event corresponding to a single BMMC cell. The expression of one of either CD33 or CD371 is shown on the y- and x-axis respectively. The plot shows the manual gating for dual positive events (grey shaded area). The gate threshold for non-specific antibody binding was calculated using the known single antigen positive cell types present. FIG. 12B is a graph that shows the percentage of cell detection events falling within the dual positive gate of FIG. 12A for each BMMC cell population identified by FlowSOM for each BMMC sample.

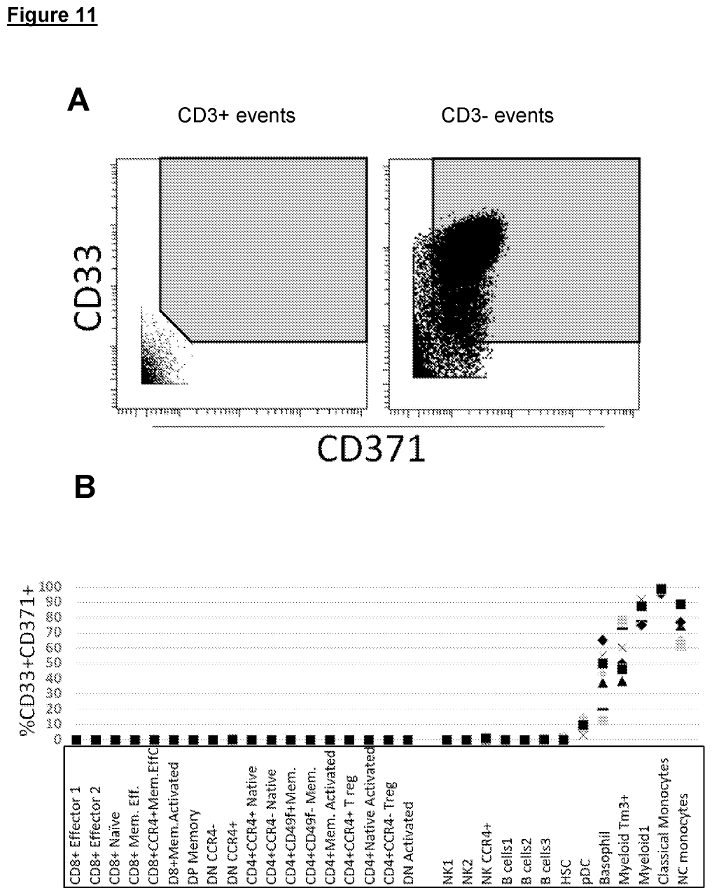

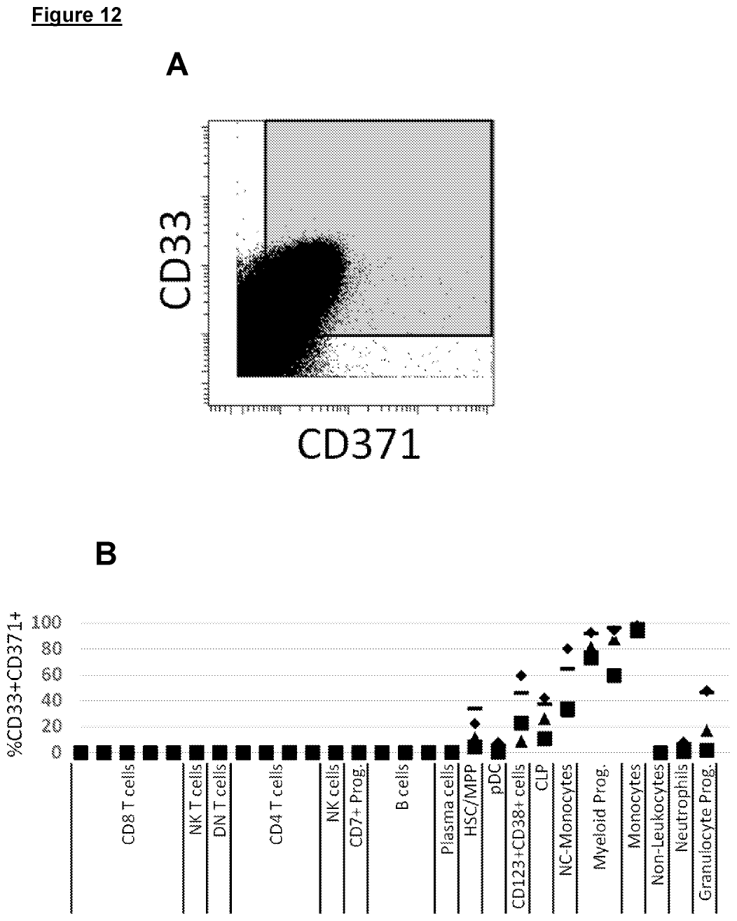

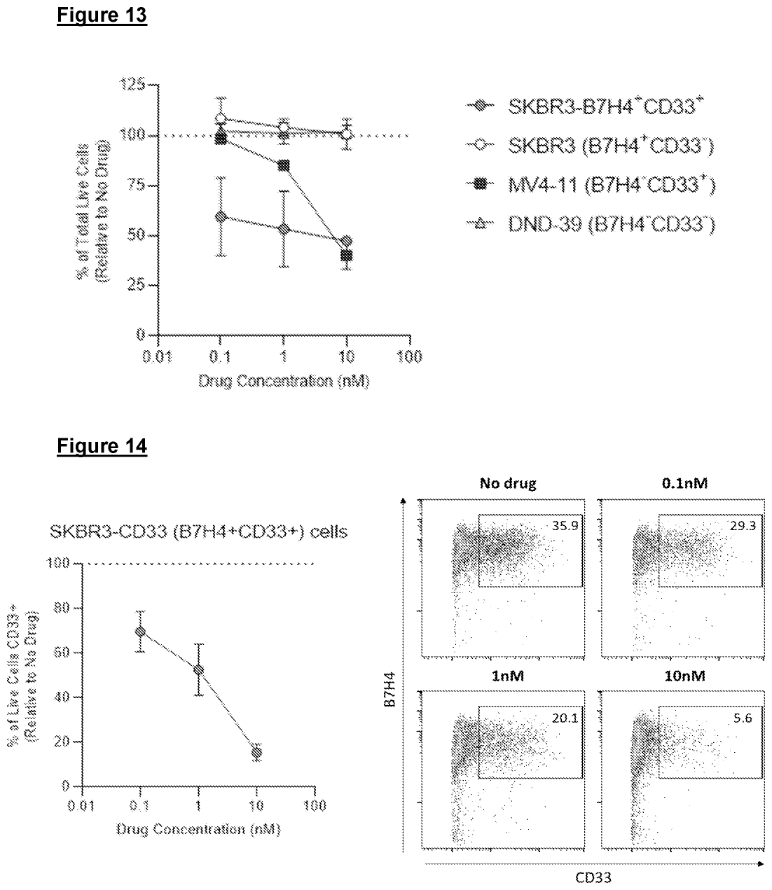

FIG. 13: Total live transfected SK-BR-3 B7H4⁺CD33⁺ cells are depleted by BVX03-a0093-AB4A-1 treatment. SK-BR-3 cells transfected with a CD33-expressing plasmid (SK-BR-3-B7H4⁺CD33⁺), untransfected SK-BR-3 cells (B7-H4⁺/CD33⁻), MV4-11 cells (B7-H4⁻/CD33⁺), and DND-39 cells (B7-H4/CD33−) were treated with BVX03-a0093-AB4A-1 at 0.1 nM, 1 nM, and 10 nM concentrations. Live cell percentage was assessed through gating on all evets/singlets/PI⁻/Annexin V⁻ and plotted as a percent of live cells in a no treatment control and error bars represent standard deviation. Dotted line represents live cells in no treatment control. n=2 biological repeats for all cell lines (2 technical repeats per biological repeat). NOTE CD33 transfection of SK-BR-3 cells was between 35-50%, so SK-BR-3-CD33 cells are a mixed population of CD33⁺ and CD33⁻ cells. Therefore, it is understandable why cell killing by BVX03-a0093-AB4A-1 is not 100% efficient.

FIG. 14: Live transfected SKBR3 (CD33⁺/B7H4⁺) cells are depleted through treatment with BVX03-a0093-AB4A-1. Percent of live B7H4⁺CD33⁺ cells in SK-BR-3-CD33 population after BVX03-a0093-AB4A-1 treatment at indicated doses. Data expressed as a percentage of B7H4⁺CD33⁺ cells in no treatment controls. Dotted line indicates B7H4⁺CD33⁺ cells in no treatment control and error bars represent standard deviation. N=2 biological repeats for all doses (2 technical repeats per biological repeat). Representative flow cytometry plots for BVX03-a0093-AB4A-1 treatments gated on B7-H4⁺CD33⁺ cells. Numbers are percentage of cells within the B7H4⁺CD33⁺ gate.

FIG. 15: Effect of CD33×B7H4 ADC on the differentiation of healthy human CD34+ progenitor cells to healthy human myeloid CD33+ cells within a colony forming unit assay. Human CD34+ progenitor cells were suspended in Methocult and IMDM media and tested against 0, 1 and 10 nM concentrations of BVX03-a0093-AB4A-1. The cells were incubated at 37° C., 5% CO₂ for 10 days. Following incubation, colony counts were performed, and the data was plotted in Excel. Average±SEM % colony counts relative to control cells only wells. n=2 biological repeats (2 technical replicates per biological repeat).

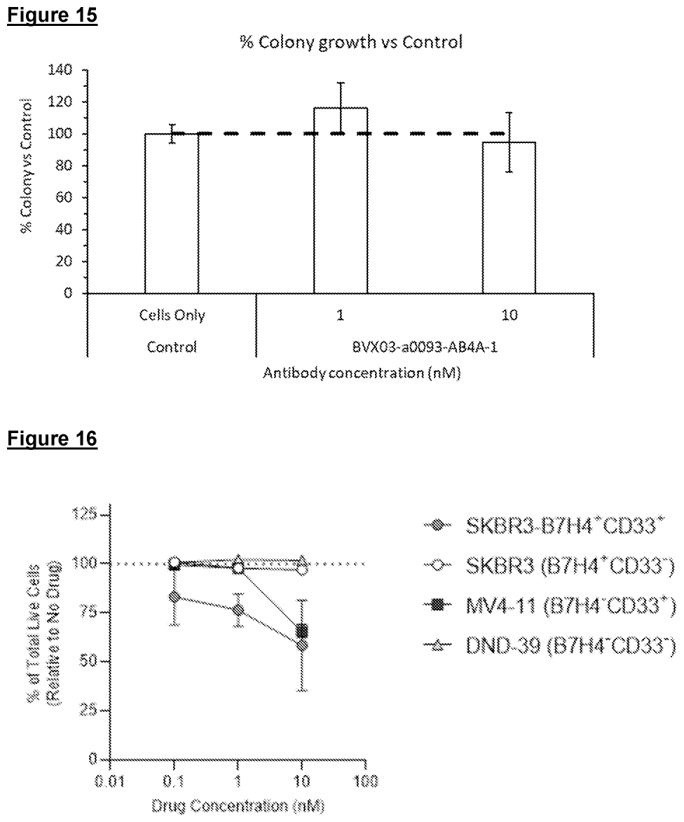

FIG. 16: Total live transfected SK-BR-3-CD33 cells are depleted by BVX03-b0096-AB6A-1 treatment. SK-BR-3 cells transfected with a CD33-expressing plasmid (SKBR3-B7H4+CD33+), untransfected SK-BR-3 cells (B7-H4+/CD33−), MV4-11 cells (B7-H4−/CD33+) and DND-39 cells (B7-H4−/CD33−) were treated with BVX03-b0096-AB6A-1 at 0.1 nM, 1 nM, and 10 nM concentrations. Live cell percentage was assessed through gating on all evets/singlets/PI-/Annexin V- and plotted as a percent of live cells in a no treatment control. Dotted line represents live cells in no treatment control and error bars represent standard deviation. n=1 biological repeat for all cell lines (2 technical repeats per biological repeat). NOTE CD33 transfection of SK-BR-3 cells was only 27%, so SK-BR-3-CD33 cells are a mixed population of CD33+ and CD33-cells. Therefore, it is understandable why cell killing by BVX06-b0096-AB6A-1 is not 100% efficient.

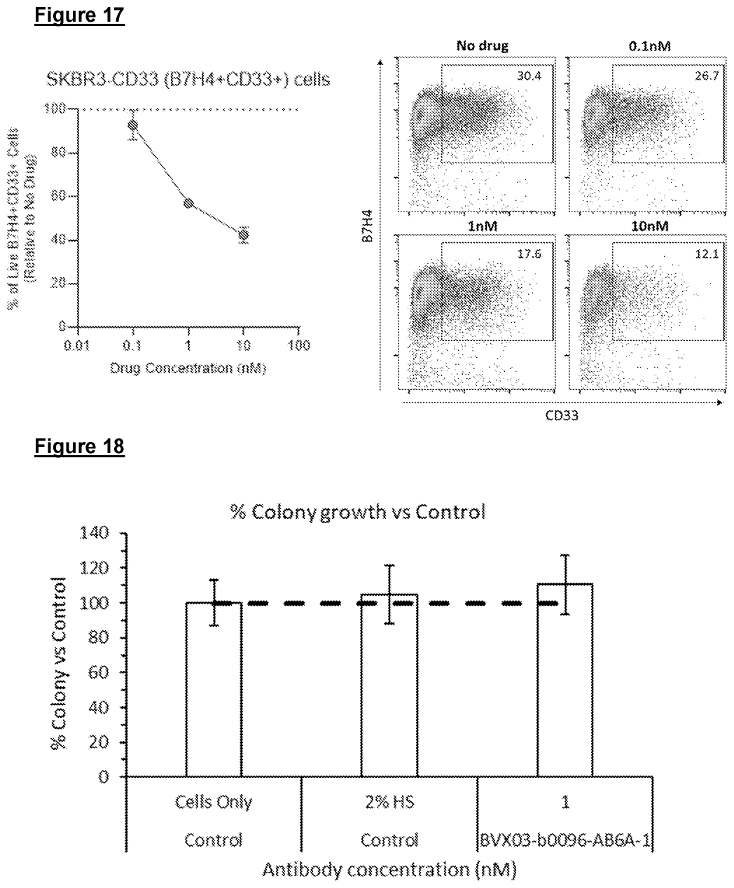

FIG. 17: Live transfected SKBR3 (CD33+/B7H4+) cells are depleted through treatment with BVX03-b0096-AB6A-1. Percent of live B7H4⁺CD33⁺ cells in SKBR3-B7H4⁺CD33⁺ population after BVX06-b0096-AB6A-1 treatment at indicated doses. Data expressed as a percentage of B7H4⁺CD33⁺ cells in no treatment controls. Dotted line indicates B7H4⁺CD33⁺ cells in no treatment control and error bars represent standard deviation. N=2 biological repeats for all doses (2 technical repeats per biological repeat). Representative flow cytometry plots for BVX03-b0096-AB6A-1 treatments gated on B7-H4⁺CD33⁺ cells. Numbers are percentage of cells within the B7H4⁺CD33⁺ gate.

FIG. 18: Effect of CD33×B7H4 ADC on the differentiation of healthy human CD34⁺ progenitor cells to healthy human myeloid CD33⁺ cells within a colony forming unit assay. Human CD34⁺ progenitor cells were suspended in Methocult and IMDM media and tested against 0 and 1 nM concentrations of BVX03-b0096-AB6A-1. The cells were incubated at 37° C., 5% CO₂ for 10 days. Following incubation, colony counts were performed, and the data was plotted in Excel. Average±SD % colony counts relative to control cells only wells. n=1 biological repeats (2 technical replicates per biological repeat).

### EXAMPLES

The invention is further illustrated in the following non-limiting examples.

### Example 1: Multivariate Analysis of 7 Healthy PBMC and 4 Healthy BMMC Patient Samples Characterises Sample Heterogeneity and Identifies Cell Types

Healthy PBMC samples collected from healthy human subjects first underwent a process to separate cells contained in the PBMC samples into cell populations which express CD3 and those which do not. CD3 is almost exclusively found expressed on the cell surface of T-cells, therefore this method separates any T-cells from other peripheral blood mononuclear cells present in the sample.

### Methods

Healthy peripheral blood mononuclear cell (PBMC; acquired from Stem cell, Cat #70025.1) samples derived from 7 separate donors were barcoded and pooled prior to staining (as detailed below), while the healthy bone marrow mononuclear cells (BMMC; acquired from Stem cell, Cat #70001.1) acquired from 4 separate donors were stained and run through a mass cytometer individually.

All centrifuge steps were performed at 500 rcf/5 mins/4° C. unless stated otherwise. 1 mL of cryopreserved cells were thawed and mixed with 1 mL of 37° C. culture media containing RPMI buffer (Sigma Aldrich; Cat #R0883) containing 10% FCS, L-Glut and penicillin/streptomycin. An additional 8 mL of 37° C. culture media was then added while agitating and cells were immediately centrifuged at room temperature (RT). Samples were resuspended in 5 ml of culture media and counted using a haemocytometer. 3×10⁶ cells were removed and placed into a separate tube for each sample to be stained for mass cytometry. These samples were washed once in ice cold MaxPAR PBS (Fluidigm; Cat #201058) and then PBMCs were resuspended in the appropriate anti-CD45 live-cell barcoding mixture (antibodies were diluted in ice cold cell staining buffer [CSB; Fluidigm, Cat #201068]; see Table 1 for antibody details and Table 2 for barcoding strategy), while BMMC were resuspended immediately in Cell-ID Cisplatin (see below). PBMC samples were barcoded on ice for 30 mins before being washed twice in ice cold CSB and washed once in ice cold PBS. During the PBS wash, samples were pooled into one tube before being centrifuged. BMMC or pooled PBMC were resuspended at 10⁷ cells/mL in a working solution (1:1000 dilution in RT MaxPAR PBS) of Cell-ID Cisplatin. Cells were left at RT for 5 mins, after which, 3× volume of CSB was added to each sample before being centrifuged. Cell pellets were then resuspended in FcX blocking solution at 50 μL/3×10⁶ cells (FcX stock diluted 1:10 dilution in CSB) and incubated at room temperature for 10 mins. A 2× concentrated antibody cocktail (see Table 2 for antibody details) was then directly added to the cells suspended in FcX solution and incubated for a further 30 mins, agitating after 15 mins. Samples were washed twice in ice cold CSB, once in ice cold PBS before being resuspended in RT 1.6% formaldehyde (1:10 dilution in MaxPAR PBS; Thermo Scientific, Cat #28906) and incubated at RT for 10 mins. Cells were then centrifuged at 800 rcf/5 mins/4° C. and resuspended at 3×10⁶ cells/mL in Intercalator solution (1:2000 dilution of 125 nM Cell-ID Intercalator-Ir [Fluidigm, Cat #201192A] in Fix and Perm buffer [Fluidigm, Cat #201067]). Cells were left overnight at 4° C.

The next day, cells were washed once in CSB and twice in CAS solution (Fluidigm, Cat #201240). Cells were filtered through a 30 UM filter-top test tube (Fisher Scientific, Cat #08-771-23), counted, and resuspended at 7.5×10⁵ cells/mL. EQ™ Four Element Calibration Beads were added 1:10 and the sample was run through a Helios mass cytometer, collecting 1 million events for each BMMC sample or 3 million events of the pooled PBMC.

TABLE 1
Mass cytometry antibody Panel

| | Antigen | | | |
|---|---|---|---|---|
| Metal | Panel | Assay Function | Company | Cat# |
| 89Y | CD45 | Barcoding/Phenotyping | Fluidigm | 3089003B |
| 106Cd | CD45 | Barcoding for PBMC | Biolegend | 304045 |
| 110Cd | CD45 | Barcoding for PBMC | Biolegend | 304045 |
| 111Cd | CD45 | Barcoding for PBMC | Biolegend | 304045 |
| 114Cd | CD45 | Barcoding for PBMC | Biolegend | 304045 |
| 115In | CD371 | Phenotyping | Biolegend | 353602 |
| 116Cd | CD45 | Barcoding for PBMC | Biolegend | 304045 |
| 141Pr | CCR6 | Phenotyping | Fluidigm | 3141014A |
| 142Nd | CD19 | Phenotyping | Fluidigm | 3142001B |
| 143Nd | CD127 | Phenotyping | Fluidigm | 3143012B |
| 144Nd | CD15 | Phenotyping | Fluidigm | 3144019B |
| 145Nd | TIM3 | Phenotyping | Biolegend | 345019 |
| 146Nd | CD7 | Phenotyping | Biolegend | 343111 |
| 147Sm | CD11c | Phenotyping | Fluidigm | 3147008B |
| 148Nd | CD16 | Phenotyping | Fluidigm | 3148004B |
| 149Sm | CCR4 | Phenotyping | Fluidigm | 3149029A |
| 150Nd | CD138 | Phenotyping | Fluidigm | 3150012B |
| 151Eu | CD123 | Phenotyping | Fluidigm | 3151001B |
| 152Sm | CD5 | Phenotyping | Biolegend | 300627 |
| 153Eu | CD33 | Phenotyping | Biolegend | 303419 |
| 154Sm | CD28 | Phenotyping | Biolegend | 302937 |
| 155Gd | CD45RA | Phenotyping | Fluidigm | 3155011B |
| 156Gd | CD10 | Phenotyping | Fluidigm | 3156001B |
| 158Gd | CD135 | Phenotyping | Fluidigm | 3158019B |
| 159Tb | CD96v2 | Phenotyping | Biolegend | 338402 |
| 160Gd | CD13 | Phenotyping | Fluidigm | 3160014B |
| 161Dy | CD90 | Phenotyping | Fluidigm | 3161009B |
| 162Dy | CD66b | Phenotyping | Fluidigm | 3162023B |
| 163Dy | CD34 | Phenotyping | Fluidigm | 3163014B |
| 164Dy | CD49f | Phenotyping | Fluidigm | 3164006B |
| 165Ho | CD45RO | Phenotyping | Fluidigm | 3165011B |
| 166Er | CD24 | Phenotyping | Fluidigm | 3166007B |
| 167Er | CCR7 | Phenotyping | Fluidigm | 3167009A |
| 168Er | CD8 | Phenotyping | Fluidigm | 3168002B |
| 169Tm | CD25 | Phenotyping | Fluidigm | 3169003B |
| 170Er | CD3 | Phenotyping | Fluidigm | 3170001B |
| 171Yb | B7H4 | Phenotyping | Biolegend | 358102 |
| 172Yb | CD38 | Phenotyping | Fluidigm | 3172007B |
| 173Yb | HLADR | Phenotyping | Fluidigm | 3173005B |
| 174Yb | CD4 | Phenotyping | Fluidigm | 3174004B |
| 175Lu | CD14 | Phenotyping | Fluidigm | 3175015B |
| 176Yb | CD56 | Phenotyping | Fluidigm | 3176008B |
| 209Bi | CD70 | Phenotyping | Biolegend | 355102 |

Samples were initially stained with 2 of the 5 possible metal-isotope conjugated anti-CD45 antibodies shown in Table 2 above, to produce a unique metal combination tag (or barcode) for each sample. Samples could then be pooled together into the same tube allowing them to be stained and run through the mass cytometer simultaneously, greatly reducing technical variability within the experiment. Events could then be separated in silico after data collection into the samples that they originated from by bivariate gating on their barcode metal signal.

### Data Analysis

Events were normalised over time against the signal on the EQ™ Four Element Calibration Beads using the CyTOF software. Normalised FCS files were then uploaded to Cytobank cloud software for analysis. For PBMC, samples were debarcoded by separating samples using bivariate Boolean gating of the barcoding metals. Cells from PBMC were also split into CD3− and CD3+ events prior to further multivariate analysis. Cells from each sample type (i.e., CD3+ PBMC, CD3− PBMC, BMMC) were clustered separately using FlowSOM, which groups cells together according to their similarities in antigen expression (termed metaclusters). After FlowSOM analysis, cells from each sample type were also passed through the viSNE algorithm that allows visualisation of the 37 phenotyping antigen dimensions in 2-dimensional space (i.e., tSNE1 and tSNE2); where the values of the 2 new tSNE parameters for each cell is equivalent to its phenotype in multidimensional space. FlowSOM metaclusters identified by FlowSOM could then be overlayed onto the tSNE plot for visualisation and given nomenclature for cell type (e.g., CD4 T cells) according to their median antigen phenotypes (FIG. 1). Cells for each cell type (i.e., FlowSOM metacluster) within each sample type were then analysed for percentage of dual antigen positivity identified using manual bivariate gating.

### Results

The tSNE plots of FIG. 1 show that a number of different PBMC cell types were identified by FlowSOM. Each of these cell types was identified using the cell surface expression of the specific marker proteins shown in Table 1. Each of the cell surface marker proteins identified is shown on the cell type lists of FIG. 1. For example, the cell population labelled “3” on the CD3+ tSNE plot corresponds to naïve T-cells expressing CD8 on the cell surface; the cell population labelled “1” on the CD3-tSNE plot corresponds to NK1 cells. The tSNE plots of FIG. 2 show that a number of different BMMC cell types were identified by FlowSOM. Each of the cell types was identified using one of the cell surface proteins as shown in Table 1. For example, the cell population labelled “15” on the BMMC tSNE plot corresponds to B cells present within the BMMC patient samples.

By separating the different PBMC and BMMC cell types using this data analysis method it was possible to investigate the overall expression pattern of specific cell surface proteins of each cell type.

### Example 2: Dual CD33 and B7H4 Cell Surface Expression on PBMC and BMMC Cell Populations

### Methods

The method used to assess the expression pattern of CD33 and B7H4 on the cell surface of both PBMC and BMMC cell populations was the same as that used in Example 2 above but with bivariate plots produced for CD3 and B7H4 on y- and x-axis respectively (FIG. 5 and FIG. 6).

### Results

FIG. 3A shows that there were few dual CD33+/B7H4+ events in both T-cell (CD3+) and non-T-cell (CD3−) PBMC cell types. FIG. 3B shows the percentage of each PBMC cell type population positive for both CD33 and B7H4. None of the PBMC cell populations contained any cells that were expressing both CD33 and B7H4 on their cell surface. FIG. 4A shows that there were almost no dual CD33+/B7H4+ detection events on BMMC cells from healthy patient samples.

These results show that bispecifically targeting CD33 and B7H4 for the treatment of solid tumour and haematological malignancies would effectively target cancer-associated myeloid-derived suppressors cells expressing both CD33 and B7H4 while avoiding targeting other healthy haematological cell populations. Using this approach, it would be possible to avoid any off-target cytotoxic effects such as immune suppression and myelosuppression that would otherwise occur when treating solid tumour or haematological malignancies by targeting a single antigen or two antigens expressed on the surface of the same healthy cell.

### Example 3: Dual CD25 and CD34 Cell Surface Expression on PBMC and BMMC Cell Populations

### Methods

The method used to assess the expression pattern of CD25 and CD34 on the cell surface of both PBMC and BMMC cell populations was the same as that used in Example 2 above but with bivariate plots produced for CD19 and CD34 on y- and x-axis respectively (FIG. 5 and FIG. 6).

### Results

FIG. 5A shows that there were dual CD25+/CD34+ events in the non-T-cell (CD3−) PBMC cell types. FIG. 5B shows the percentage of each PBMC cell type population positive for both CD25 and CD34. FIG. 5B shows that a high percentage of the haematopoietic stem cell (HSC) population identified in Example 1 demonstrate dual expression of both CD25 and CD34. Therefore, any composition that targets both CD25 and CD34 as a treatment for a malignancy would also target HSCs expressing CD25 and CD34. Off-target toxicity to HSCs caused by a treatment targeting CD25 and CD34 would lead to direct myelosuppression in bone marrow, an unwanted and potentially life-threatening side effect of treatment. FIG. 6A shows that there were few dual CD25+/CD34+ detection events on BMMC cells from healthy patient samples. Despite this, any treatment that targets both CD25 and CD34 does not avoid negative off-target cytotoxicity and would lead to myelosuppression by simultaneously targeting HSCs and any malignancy.

### Example 4: Dual CD56 and CD7 Cell Surface Expression on PBMC and BMMC Cell Populations

### Methods

The method used to assess the expression pattern of CD56 and CD7 on the cell surface of both PBMC and BMMC cell populations was the same as that used in Example 2 above but with bivariate plots produced for CD56 and CD7 on y- and x-axis respectively (FIG. 7 and FIG. 8).

### Results

FIG. 7A shows that overall there were a substantial number of dual CD56+/CD7+ events in non-T-cell (CD3−) PBMC cell types. FIG. 7B shows the percentage of each PBMC cell type population positive for both CD56 and CD7. FIG. 7B shows that a high percentage of the Natural Killer 1 (NK1), Natural Killer 2 (NK2) and Natural Killer CCR4+ (NK CCR4+) cell populations identified in Example 1 demonstrate dual expression of both CD56 and CD7 and would therefore experience off-target cytotoxicity caused by a treatment targeting both CD56 and CD7. Targeting NK cells would lead to reduced innate immunity thereby making the patient susceptible to infection and disease. Furthermore, FIG. 8A shows that there were a substantial number of dual CD56+/CD7+ detection events in BMMC cell populations from healthy patient samples. FIG. 8B shows that the majority of the detection events arise from the CD7+ Progenitor cell type. Any off-target cytotoxicity directed towards this cell type leads to immune suppression and potential myelosuppression. Therefore, any treatment that targets both CD56 and CD7 does not avoid negative off-target cytotoxicity and would lead to immune suppression by simultaneously targeting NK cells and BMMCs along with any malignancy.

### Example 5: Dual CD56 and CD11c Cell Surface Expression on PBMC and BMMC Cell Populations

### Methods

The method used to assess the expression pattern of CD56 and CD11c on the cell surface of both PBMC and BMMC cell populations was the same as that used in Example 2 above but with bivariate plots produced for CD56 and CD11c on y- and x-axis respectively (FIG. 9 and FIG. 10).

### Results

FIG. 9A shows that overall there were a substantial number of dual CD56+/CD11c+ events in non-T-cell (CD3−) PBMC cell types. FIG. 9B shows the percentage of each PBMC cell type population positive for both CD56 and CD11c. FIG. 9B shows that a high percentage of the Natural Killer 1 (NK1), Natural Killer 2 (NK2) and Natural Killer CCR4+ (NK CCR4+), Myeloid Tlm3 and Myeloid 1 cell populations identified in Example 1 demonstrate dual expression of both CD56 and CD11c and would therefore experience off-target cytotoxicity caused by a treatment targeting both CD56 and CD11c. Targeting NK cells would lead to reduced innate immunity thereby making the patient susceptible to infection and disease. Targeting Myeloid cell populations would lead to myelosuppression. Furthermore, FIG. 10A shows that there were a substantial number of dual CD56+/CD11c+ detection events in BMMC cell populations from healthy patient samples. FIG. 10B shows that the majority of the detection events arise from the CD7+ Progenitor cell type. Any off-target cytotoxicity directed towards this cell type leads to immune suppression and potential myelosuppression. Therefore, any treatment that targets both CD56 and CD11c does not avoid negative off-target cytotoxicity and would lead to immune suppression by simultaneously targeting NK cells, myeloid cells and BMMCs along with any malignancy.

### Example 6: Dual CD33 and CD371 Cell Surface Expression on PBMC and BMMC Cell Populations

### Methods

The method used to assess the expression pattern of CD33 and CD371 on the cell surface of both PBMC and BMMC cell populations was the same as that used in Example 2 above but with bivariate plots produced for CD33 and CD371 on y- and x-axis respectively (FIG. 11 and FIG. 12).

### Results

FIG. 11A shows that overall there were a substantial number of dual CD33+/CD371 events in non-T-cell (CD3−) PBMC cell types. FIG. 11B shows the percentage of each PBMC cell type population positive for both CD33 and CD371. FIG. 11B shows that a large percentage of Basophils, Myeloid cells and Monocyte cell populations identified in Example 1 demonstrate dual expression of both CD33 and CD371 and would therefore experience off-target cytotoxicity caused by a treatment targeting both CD33 and CD371. Targeting Myeloid cell populations would lead to immune suppression and specifically myelosuppression. Targeting Monocytes and Basophils would lead to reduced immunity thereby causing the patient to be more susceptible to infection and disease during the course of any treatment. Furthermore, FIG. 12A shows that there were a substantial number of dual CD33+/CD371+ detection events in BMMC cell populations from healthy patient samples. A high percentage of Myeloid progenitor cells, monocytes, Common Lymphoid Progenitor cells (CLP) and CD123+/CD38+ cells isolated from healthy patient BMMC samples demonstrate dual expression of both CD33 and CD371. Any off-target cytotoxicity directed towards these cell types leads to immune suppression and specifically myelosuppression. Therefore, any treatment that targets both CD33 and CD371 does not avoid negative off-target cytotoxicity and would lead to immune suppression and myelosuppression by simultaneously targeting BMMC cells along with any malignancy.

### Summary

The expression of certain cell surface protein pairs on cancerous cells allows the effective targeting of the cancerous cells using a cytotoxic composition that binds to both proteins of such a protein pair. However, the finding that a number of these protein pairs are also expressed on certain healthy haematological cells (PBMC and BMMC cells) shows that it is not possible to easily determine which cell surface protein pairs would avoid any off-target cytotoxicity. Off-target cytotoxicity that targets healthy PBMC and/or BMMC cells leads to immune suppression and/or myelosuppression and/or impaired immune function which are common side effects of anti-cancer chemotherapy. Targeting a protein pair in which both cell surface proteins are expressed on cancerous cells but both cell surface proteins are not co-expressed together on healthy haematological cells avoids any off-target cytotoxicity of these healthy cells. This reduces immune suppression and/or myelosuppression and/or impairment of immune function.

The experiments show that cell inhibiting agents targeting malignant cells expressing CD33 and B7H4 could prove useful treatments as they would be non-immune suppressing. This is particularly advantageous for antibody-based therapies where impaired immune function is often a major factor. In contrast, the experiments also indicated that cell inhibiting agents targeting malignant cells expressing CD25 and CD34; or CD56 and CD7; or CD56 and CD11c; or CD33 and CD371 would not be suitable as treatments for targeting malignant cells expressing those antigens as such targeting would result in immune suppression and/or mylosuppression and/or impaired immune function.

Using the methods detailed above it was possible to determine which cell surface protein pairs are expressed on cancerous cells but not healthy PBMC or BMMC cells isolated from healthy human patients.

It is also possible to use method to screen patients with a confirmed malignancy to determine whether their healthy haematological cells co-express both cell surface proteins that are targeted by a specific composition designed to target a protein pair expressed on the cell surface of a cancerous cell.

The forgoing embodiments are not intended to limit the scope of the protection afforded by the claims, but rather to describe examples of how the invention may be put into practice.

### Example 7: CD33×B7H4 Bispecific Antibodies Demonstrate Selective Cell Killing Fab Expression

### Sequences for Fab Expression:

Gemtuzumab VH-antiCD33:
(SEQ ID NO. 3)
EVQLVQSGAEVKKPGSSVKVSCKASGYTITDSNIHWVRQAPGQSLEWIG
YIYPYNGGTDYNQKFKNRATLTVDNPTNTAYMELSSLRSEDTAFYYCVN
GNPWLAYWGQGTLVTVSS
Gemtuzumab VL-antiCD33:
(SEQ ID NO. 4)
DIQLTQSPSTLSASVGDRVTIICRASESLDNYGIRFLTWFQQKPGKAPK
LLMYAASNQGSGVPSRFSGSGSGTEFTLTISSLQPDDFATYYCQQTKEV
PWSFGQGTKLEIK
VL2B-antiB7H4:
(SEQ ID NO. 5)
DIVMTQTPLSLPVTLGQPASISCRSSQSLVHINGNTYLHWYQQRPGQSP
RVLIYKVSNRFSGVPDRFSGSGSGTDFTLKISRVEAEDVGVYFCSQSTH
VPLTFGQGTKVEIK
VH1C-antiB7H4:
(SEQ ID NO. 6)
EVQLVQSGAEVKKPGASVKVSCKASGYTFTDYYMNWVRQAPGQGLEWIG
IINPYNGDTSYNQKFKGRVTLTVDKSTSTAYMELSSLRSEDTAVYYCAR
YPESTYWGQGTLVTVSS

VH and VL fragments were cloned into separate mammalian expression vectors encoding for human C_{H}1 and CL domains downstream, respectively. Transfection grade DNA was prepared using the Plasmid Plus Midi Kit (Qiagen, Cat. No. 12945) according to manufacturer's instructions.

Fabs were expressed using the Expi293F (LifeTech, Cat. No. A14525) expression system following manufacturer's instructions, and subsequently batch purified using anti-C_{H}1 resin.

Briefly, for each Fab, 100 ml of culture was transfected, incubated at 37° C., 8% CO₂, 80% humidity, and harvested by centrifugation after 6 days. The supernatant was filtered using a 0.22 μM filter (Merck, Cat. No. 15939180) and stored at 4° C. until required. 10×PBS (ThermoFisher, Cat. No. 70013032) was added at 1/10th volume of the supernatant. CaptureSelect™ CH1-XL Affinity Matrix (ThermoFisher, Cat. No. 1943462005), an anti-CH1 resin, was added in an appropriate volume. The tubes were incubated overnight at 4° C. on a rotating wheel to ensure thorough mixing of the supernatant and resin. The following day the tubes were centrifuged, and the resin transferred to a Proteus ‘1-Step Batch’ Midi Spin Column (ProteinArk, Cat. No. GEN-1SB08) followed by two wash steps using wash buffer (1×PBS with 200 mM NaCl). The protein was eluted using 600 μL 0.2M Glycine pH3.0, directly neutralised in 200 μL of 1M Tris-HCl pH8.0. The protein concentration was measured by A280 reading before dialysis into 1×PBS overnight using GeBAFlex Midi Tubes, 8 kDa Cut-Off (ProteinArk, Cat. No. MD6-22-30). The protein concentrations were re-measured, quality assessed by SDS-PAGE, and subsequently stored at 4° C. ready for Bi-fab formation.

### Fab-scFv-Fc (V Format) Expression and Purification

### Sequences for Fab-scFv-Fc Expression:

Gemtuzumab VH antiCD33:
(SEQ ID NO. 7)
EVQLVQSGAEVKKPGSSVKVSCKASGYTITDSNIHWVRQAPGQSLEWIG
YIYPYNGGTDYNQKFKNRATLTVDNPTNTAYMELSSLRSEDTAFYYCVN
GNPWLAYWGQGTLVTVSS
Gemtuzumab VL-antiCD33:
(SEQ ID NO. 8)
DIQLTQSPSTLSASVGDRVTIICRASESLDNYGIRFLTWFQQKPGKAPK
LLMYAASNQGSGVPSRFSGSGSGTEFTLTISSLQPDDFATYYCQQTKEV
PWSFGQGTKLEIK
VL2B_VH1C scFv-antiB7H4:
(SEQ ID NO. 9)
DIVMTQTPLSLPVTLGQPASISCRSSQSLVHINGNTYLHWYQQRPGQSP
RVLIYKVSNRFSGVPDRFSGSGSGTDFTLKISRVEAEDVGVYFCSQSTH
VPLTFGQGTKVEIKGGGGSGGGGSGGGGSGGGGSEVQLVQSGAEVKKPG
ASVKVSCKASGYTFTDYYMNWVRQAPGQGLEWIGIINPYNGDTSYNQKF
KGRVTLTVDKSTSTAYMELSSLRSEDTAVYYCARYPESTYWGQGTLVTV
SS
Knob: CH1, CH2 & CH3
(SEQ ID NO. 10)
TKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVS
LWCLVKGFYPSDIAVEWESNGQPENNYKTTPPVLDSDGSFFLYSKLTVD
KSRWQQGNVFSCSVMHEALHNHYTQKSLSLSPGK
Hole: G4S Linker, CH2 & CH3
(SEQ ID NO. 11)
GGGGSEPKSQDKTHTCPPCPAPELLGGPSVFLFPPKPKDTLMISRTPEV
TCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDE
LTKNQVSLSCAVKGFYPSDIAVEWESNGQPENNYKTTPPVLDSDGSFFL
VSKLTVDKSRWQQGNVFSCSVMHEALHNHYTQKSLSLSPGK

The scFv fragment for the anti-B7H4 antibody was cloned into a mammalian expression vector encoding the Heavy Chain (as the Hole part of the Knob-in-Hole technology). Transfection grade DNA was prepared using the Plasmid Plus Midi Kit (Qiagen, Cat. No. 12945) according to manufacturer's instructions. This was paired with DNA for two further vectors encoding the anti-CD33 Light Chain and the Heavy Chain (as the Knob part).

The Knob-In-Hole format version of the antibody was transfected and purified as described for Fabs. The only change was the use of Fastback Protein A Sepharose Resin 100 mL (Generon, Cat. No. NB-45-00036-25) in place of anti-CH1 resin. All other aspects were as described in the Fab transfection and purification. The protein concentrations were re-measured, and quality assessed by SDS-PAGE. It was determined that further polishing was required. This was performed by Ion Exchange Chromatography.

### Bi-Fab Chemistry and Conjugation

Fabs were modified to permit Bi-fab formation by bio-orthogonal reactive partners, this was undertaken using a similar method as described in ‘The renaissance of chemically generated bispecific antibodies, Szij P, Chudasama V, Nature Reviews Chemistry, (2021), 78-92, 5 (2)’. On formation of the Bi-Fab, the molecule was conjugated with mcMMAF targeting an average DAR 4, a similar method was used to as described in ‘Committee for Medicinal Products for Human Use (CHMP) Assessment report BLENREP’, EMA/CHMP/414341/2020 Corr.

V format molecules were reduced with DTT and conjugated with mcMMAF targeting an average DAR 6, using a similar method to as described in ‘Committee for Medicinal Products for Human Use (CHMP) Assessment report BLENREP’, EMA/CHMP/414341/2020 Corr.

CD33×B7H4 Bi-Fab ADC Demonstrates Selective and Efficient Cell Killing in Double Antigen Positive (CD33⁺B7H4⁺) Vs Single Antigen Positive Cells in Cytotoxicity Assay

Reagents

| | |
|---|---|
| BVX03-a0093-AB4A-1 (CD33 × B7H4 | In House |
| Bi-Fab ADC) | |
| SK-BR-3 | DSMZ |
| McCoys Media | Gibco |
| pCMV-CD33 Expression Plasmid | Sino Biological HG12238-UT |
| Lipofectamine 3000 Transfection | Invitrogen L3000008 |
| Reagent | |
| Opti-MEM I Reduced Serum Medium | Gibco 31985062 |
| Anti-B7-H4 APC Antibody | Biolegend #358107 |
| Anti-CD33 Fluorescein Antibody | Absolute Antibodies |
| | Ab00283-1.1 |
| Annexin V Pacific Blue | ThermoFisher Scientific A35122 |
| Annexin binding buffer | eBioscience BMS500BB |
| Propidium Iodide | Invitrogen P1304MP |
| Eppendorf tubes | |
| PBS | |
| FACS buffer (PBS w/0.1% BSA) | |

### Method

SK-BR-3 cells (B7H4⁺) were transiently transfected with an expression plasmid containing the human CD33 cDNA (HG12238-UT) using Lipofectimine 3000 in order to establish a model for our B7-H4⁺ CD33⁺ dual-positive cell line. 8.5×10⁵ cells/well of SK-BR-3 were seeded into 12 well plates and placed in a 37° C., 5% CO2 incubator for 24 hours to allow the cells to adhere. For each transfection, 50 μl Opti-MEM I mudium was mixed with 1 μl Lipofectamine 3000 and incubated for 5 minutes at room temperature. 1 μg of CD33 plasmid was mixed with 50 μl Opti-MEM I and 2 μl P3000 reagent and, subsequently, added to the Lipofectamine 3000 mix and incubed for 15 minutes. 100 μl of this DNA-Lipofectamine 3000 mix was added directly onto SK-BR-3 cells and 1 ml McCoys media (20% FCS) added to each well. Transfection efficiency was assessed after 72 hours by flow cytometry.

72 hours after transfection, 0.1, 1 or 10 nM of ADC (BVX03-a0093-AB4A-1) was added to each well of transfected SK-BR-3 B7H4⁺CD33⁺ cells. In separate 12 well plates, untransfected SK-BR-3 cells (B7-H4⁺/CD33⁻) seeded previously, MV4-11 cells (B7-H4⁻/CD33⁺, 4×10⁵ cells/well) and DND-39 cells (B7-H4⁻/CD33, 1×10⁵ cells/well) were treated with the same concentrations of ADC. After 48 hours of ADC exposure cells were harvested for flow cytometry analysis. Media was reserved from each well and the wells were washed with PBS, 0.05% trypsin was added to the wells and the cells were detached. 10% FBS contained media was used to neutralise the trypsin and each condition was placed in an Eppendorf. Cell suspensions were centriguged 300 g for 5 minutes and then washed with 0.1% BSA in PBS and transferred into eppendorfs. They were spin at 500 g for 2 minutes, supernatants were removed, and cell pellets were resuspended in antibody mix (1:100 Anti-B7H4/1:200 Anti-CD33) and incubated for 30 mins at room temperature in the dark. After incubation, cell suspensions were centrifuged 300 g for 5 minutes and then washed using 0.1% BSA in PBS. Then, the buffer was exchanged to 1× Annexin binding buffer. Spin at 500 g for 2 minutes removed supernatant and resuspend cell pellet in Annexin V mix (5 μl/100 μl Annexin Buffer) and incubate for 15 mins at room temperature in the dark. After incubation, cells were washed in annexin buffer as before and incubated with propidium iodide (5 μl/200 μl Annexin Buffer) for 10 minutes at room temperature.

Cell kill was assessed by using flow cytometry (MACSQuant16) and data analysed using FlowJo™ v10.8 Software (BD Life Sciences).

No reduction in healthy human CD33⁺ myeloid colonies seen in Colony Forming Unit Granulocyte-Macrophage (CFU-GM) assay using CD33×B7H4 Bi-Fab ADC

Reagents

| | |
|---|---|
| BVX03-a0093-AB4A-1 (CD33 × B7H4 | In House |
| Bi-Fab ADC) | |
| Human Cord Blood CD34+ Cells | Stemcell Technologies #70008.1 |
| (mixed donor) | |
| MethoCult ™ Optimum Without EPO | Stemcell Technologies #04437 |
| Iscove's MDM with 2% FBS | Stemcell Technologies #7700 |
| SmartDish | Stemcell Technologies #27370 |
| STEMgrid ™-6 | Stemcell Technologies #27000 |

### Method

Methocult was thawed at 4° C. overnight. Methocult was shaken and allowed to stand at room temperature until bubbles had dispersed and aliquoted into 3 ml aliquots. Concentrations of 1 nM and 10 nM were prepared for each ADC (BVX03-a0093-AB4A-1) in PBS. Each concentration was tested across duplicate wells. CD34+ cells were thawed at 37° C. and suspended in 2% FBS IMDM. 270 μl of suspension containing 3000 CD34+ cells were pipetted into each 1.5 ml tube containing 30 μl ADC and the solution was gently mixed by pipetting. The cell suspension/ADC mix was pipetted into a 3 ml aliquot of Methocult, vortexed for 5 seconds and allowed to stand at room temperature until the bubbles had disappeared. Using an 18-gauge blunt needle and 6 ml syringe, 1 ml of cell/ADC/Methocult mix was transferred to a well of a Smart dish. Each plate was rocked to ensure the Methocult covered the entire well surface evenly and then placed in a 37° C., 5% CO₂ incubator. The colonies were counted on day 10 and the data plotted in Excel.

CD33×B7H4 Bi-Fab ADC selectively kills dual positive (CD33⁺B7H4⁺) cells compared to single antigen positive or double antigen negative cells. The CD33×B7H4 Bi-Fab ADC does not cause any decrease in colony formation of CD33⁺ healthy human myeloid cells, derived from healthy human cord blood donors, within CFU-GM assay (industry standard assay for assessing risk for myelosuppression in the clinic).

CD33×B7H4 V format ADC demonstrates selective and efficient cell killing in double antigen positive (CD33⁺B7H4⁺) vs single antigen positive cells in cytotoxicity assay

Reagents

| | |
|---|---|
| BVX03-b0096-AB6A-1 (CD33 × B7H4 | In House |
| V format ADC) | |
| SK-BR-3 | DSMZ |
| McCoys Media | Gibco |
| pCMV-CD33 Expression Plasmid | Sino Biological HG12238-UT |
| Lipofectamine 3000 Transfection | Invitrogen L3000008 |
| Reagent | |
| Opti-MEM I Reduced Serum Medium | Gibco 31985062 |
| Anti-B7-H4 APC Antibody | Biolegend #358107 |
| Anti-CD33 Fluorescein Antibody | Absolute Antibodies |
| | Ab00283-1.1 |
| Annexin V Pacific Blue | ThermoFisher Scientific A35122 |
| Annexin binding buffer | eBioscience BMS500BB |
| Propidium Iodide | Invitrogen P1304MP |
| Eppendorf tubes | |
| PBS | |
| FACS buffer (PBS w/0.1% BSA) | |

### Method

SK-BR-3 cells (B7H4⁺) were transiently transfected with an expression plasmid containing the human CD33 cDNA (HG12238-UT) using Lipofectimine 3000 in order to establish a model for our CD33⁺/B7H4⁺ dual-positive cell line. 8.5×10⁵ cells/well of SK-BR-3 were seeded into 12 well plates and placed in a 37° C., 5% CO2 incubator for 24 hours to allow the cells to adhere. Separate plates of SK-BR-3 cells were seeded for comparison to transfected cell in the cell kill assay. For each transfection, 50 μl Opti-MEM I medium was mixed with 1 μl Lipofectamine 3000 and incubated for 5 minutes at room temperature. 1 μg of CD33 plasmid was mixed with 50 μl Opti-MEM I and 2 μl P3000 reagent and, subsequently, added to the Lipofectamine 3000 mix and incubed for 15 minutes. 100 μl of this DNA-Lipofectamine 3000 mix was added directly onto SKBR3 cells and 1 ml McCoys media (20% FCS) added to each well. Transfection efficiency was assessed after 72 hours by flow cytometry.

72 hours after transfection, 0.1, 1 or 10 nM of ADC was added to each well of transfected SKBR3 cells. In separate 12 well plates, untransfected SKBR3 cells (B7-H4⁺/CD33⁻), seeded previously, and MV-411 cells (B7-H4/CD33⁺, 4×10⁵ cells/well) were treated with the same concentrations of ADC. After 48 hours of ADC exposure cells were harvested for flow cytometry analysis. Media was reserved from each well and the wells were washed with PBS, 0.05% trypsin was added to the wells and the cells were detached. 10% FBS contained media was used to neutralise the trypsin and each condition was placed in an Eppendorf. Cell suspensions were centruged 300g for 5 minutes and then washed with 0.1% BSA in PBS and transferred into eppendorfs. They were spin at 500 g for 2 minutes, supernatants were removed, and cell pellets were resuspended in antibody mix (1:100 Anti-B7H4/1:200 Anti-CD33) and incubated for 30 mins at room temperature in the dark. After incubation, cell suspensions were centrifuged 300 g for 5 minutes and then washed using 0.1% BSA in PBS. Then, the buffer was exchanged to 1× Annexin binding buffer. Spin at 500 g for 2 minutes removed supernatant and resuspend cell pellet in Annexin V mix (5 μl/100 μl Annexin Buffer) and incubate for 15 mins at room temperature in the dark. After incubation, cells were washed in annexin buffer as before and incubated with propidium iodide (5 μl/200 μl Annexin Buffer) for 10 minutes at room temperature.

Cell kill was assessed by flow cytometry (MACSQuant16) and data analysed using FlowJo™ v10.8 Software (BD Life Sciences).

No reduction in healthy human CD33⁺ myeloid colonies seen in Colony Forming Unit Granulocyte-Macrophage (CFU-GM) assay using CD33×B7H4 V format ADC

Reagents

| | |
|---|---|
| BVX03-b0096-AB6A-1 (CD33 × B7H4 | In House |
| V format ADC) | |
| Human Cord Blood CD34+ Cells | Stemcell Technologies #70008.1 |
| (mixed donor) | |
| MethoCult ™ Optimum Without EPO | Stemcell Technologies #04437 |
| Iscove's MDM with 2% FBS | Stemcell Technologies #7700 |
| SmartDish | Stemcell Technologies #27370 |
| STEMgrid ™-6 | Stemcell Technologies #27000 |
| Human Serum | Merck #H3667-20ML |

### Method

Methocult was thawed at 4° C. overnight. Methocult was shaken and allowed to stand at room temperature until bubbles had dispersed and aliquoted into 3 ml aliquots. 1 nM of ADC was prepared (BVX03-b0096-AB6A-1) in PBS and tested across duplicate wells. CD34⁺ cells were thawed at 37° C. and suspended in 2% FBS IMDM. 270 μl of suspension containing 3000 CD34⁺ cells were pipetted into each 1.5 ml tube containing 30 μl ADC and the solution was gently mixed by pipetting. The media was supplemented with 2% human serum to avoid any non-specific uptake of the ADC via Fc receptor internalisation. The cell suspension/ADC mix was pipetted into a 3 ml aliquot of Methocult, vortexed for 5 seconds and allowed to stand at room temperature until the bubbles had disappeared. Using an 18-gauge blunt needle and 6 ml syringe, 1 ml of cell/ADC/Methocult mix was transferred to a well of a Smart dish. Each plate was rocked to ensure the Methocult covered the entire well surface evenly and then placed in a 37° C., 5% CO2 incubator. The colonies were counted on day 10 and the data plotted in Excel.

CD33×B7H4 V format ADC selectively kills dual positive (CD33⁺B7H4⁺) cells compared to single antigen positive or double antigen negative cells. The CD33×B7H4 V format ADC does not cause any decrease in colony formation of CD33⁺ healthy human myeloid cells, derived from healthy human cord blood donors, within CFU-GM assay (industry standard assay for assessing risk for myelosuppression in the clinic).

### SUMMARY OF EXAMPLES

The expression of specific cell surface protein pairs on cancerous cells allows the effective targeting of the cancerous cells using a cytotoxic composition that binds to both proteins of such a protein pair. However, the finding that a number of these protein pairs are also expressed on certain healthy cells (e.g. PBMC and BMMC cells) shows that it is not possible to easily determine which cell surface protein pairs would avoid any off-target cytotoxicity. Off-target cytotoxicity that targets healthy cells leads to immune suppression and/or myelosuppression and/or impaired immune function which are common side effects of anti-cancer chemotherapy. Targeting a protein pair in which both cell surface proteins are expressed on cells associated with but both cell surface proteins are not expressed on healthy cells avoids any off-target cytotoxicity of these healthy cells. This reduces immune suppression and/or myelosuppression and/or impairment of immune function.

The experiments indicated that cell inhibiting agents targeting malignant cells expressing CD33 and B7H4 could prove useful treatments as they would be non-myelosuppressing and/or non-immune suppressing. This is particularly advantageous for antibody-based therapies where myelosuppression and/or impaired immune function is often a major factor. In contrast, the experiments also indicated that cell inhibiting agents targeting malignant cells expressing CD25 and CD34; or CD56 and CD7; or CD56 and CD11c; or CD33 and CD371 would not be suitable as treatments for targeting malignant cells expressing those antigens as such targeting would result in immune suppression and/or myelosuppression and/or impaired immune function.

Using the methods detailed above it was possible to determine which cell surface protein pairs are tumour associated cells but not healthy PBMC or BMMC cells isolated from healthy human patients. Having shown the cell surface protein expression pattern a conjugated bispecific antibody targeting CD33+/B7H4+ showed preferential cytotoxicity for cells expressing both CD33 and B7H4 over cells expressing neither of these cell surface proteins or cells expressing only one of these proteins.

A conjugated antibody targeting both CD33 and B7H4 also reduced the off-target cytotoxicity observed in a CD34+ to B7H4+ myeloid differentiation colony forming assay compared to a conjugated antibody that targets a single antigen. This provides further evidence that a composition, e.g. bispecific antibody, targeting both CD33 and B7H4 would avoid any off-target immune and/or myelosuppression in a patient receiving this composition.

It is also possible to use the methods above to screen patients with a confirmed malignancy to determine whether their healthy cells express both cell surface proteins that are targeted by a specific composition designed to target a protein pair expressed on the cell surface of a cancerous cell.

The forgoing embodiments are not intended to limit the scope of the protection afforded by the claims, but rather to describe examples of how the invention may be put into practice.

1. A composition for use in the treatment of a malignancy, wherein the composition comprises an agent that binds to CD33 and B7H4.

2. The composition for use according to claim 1, wherein the composition is a non-immune suppressing treatment.

3. The composition for use according to claim 2, wherein the non-immune suppressing treatment is non-myelosuppressing treatment.

4. The composition for use according to any preceding claim, wherein the agent is an antibody or antigen binding fragment thereof.

5. The composition for use according to any preceding claim, wherein the agent is a bispecific antibody or antigen binding fragment thereof that binds CD33 and B7H4.

6. The composition for use according to a preceding claim, wherein the composition further comprises a payload.

7. The composition for use according to claim 6 wherein the payload is a cell killing agent, an immune-modulating payload, a macrophage class switching agent, or a light activatable payload.

8. The composition for use according to claim 7 wherein the immune-modulating payload is a STING agonist or a toll-like receptor agonist.

9. The composition for use according to claim 7, wherein the cell killing agent comprises a cytotoxin.

10. The composition for use according to claim 9, wherein said cytotoxin is selected from:
i) a peptide toxin; or
ii) a chemical toxin.

11. The composition for use according to claims 9 to 10, wherein the composition further comprises a linker for linking the payload to the agent that binds to CD33 and B7H4 expressed on the cell surface.

12. The composition for use according to claims 1 to 11, wherein the composition is a bispecific antibody drug conjugate.

13. The composition for use according to claims 1 to 12, wherein the malignancy comprises a malignant disease associated with an increase in myeloid derived suppressor cells.

14. The composition for use according to claims 1 to 13, wherein the malignancy is an ovarian cancer, an ovarian cancer derived cancer, Acute Myeloid Leukaemia (AML) or AML derived cancer.

15. The composition for use according to claims 1 to 13, wherein the malignancy is selected from one of the following cancers: haematological cancers including Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukaemia (CLL), Myelodysplastic Syndrome (MDS), and Diffuse Large B cell Lymphoma (DLBCL); or Hepatocellular Carcinoma; or Gynaecological Cancers including Ovarian Cancer, Cervical Cancer, and Endometrial Cancer; lung cancer, bladder cancer breast cancer, renal cell carcinoma, melanoma, colorectal cancer, gastric cancer, pancreatic cancer, thyroid cancer, liver cancer and glioblastoma.

16. A bispecific antibody or antibody fragment capable of binding CD33 and B7H4 for use in the treatment of cancer.

17. The bispecific antibody according to claim 16 wherein the cancer is a haematological cancer or Multiple Myeloma.

18. The bispecific antibody according to claim 16 or 17 wherein said use comprises contacting cells that express both CD33 and B7H4.

19. A method for treating a malignancy comprising administering to a subject in need thereof an agent that binds to CD33 and B7H4.

20. The method according to claim 19, wherein the treatment is a non-immune suppressing treatment.

21. The method according to claim 20, wherein the treatment is a non-myelosuppressing treatment.

22. The method according to any of claims 19 to 21, wherein the agent is an antibody or antigen binding fragment thereof.

23. The method according to claim 22, wherein the agent is a bispecific antibody or antigen binding fragment thereof that binds CD33 and B7H4.

24. The method according to any of claims 19 to 22, wherein the composition further comprises a payload.

25. The composition for use according to claim 24 wherein the payload is a cell killing agent, an immune-modulating payload, a macrophage class switching agent, or a light activatable payload.

26. The composition for use according to claim 25 wherein the immune-modulating payload is a STING agonist or a toll-like receptor agonist.

27. The method according to claim 25, wherein the cell killing agent comprises a cytotoxin.

28. The method according to claim 27, wherein said cytotoxin is selected from:
i) a peptide toxin; or
ii) a chemical toxin.

29. The method according to any of claims 26 to 28, wherein the composition further comprises a linker for linking the payload to the agent that binds to CD33 and B7H4.

30. The method according to any of claims 19 to 29, wherein the composition is a bispecific antibody drug conjugate.

31. The method according to claims 19 to 30, wherein the malignancy is selected from one of the following cancers: haematological cancers or Multiple Myeloma.

32. The method according to any of claims 19 to 30, wherein the malignancy is an AML or AML derived cancer.

33. The method according to any of claims 19 to 30, wherein the malignancy is selected from one of the following cancers: haematological cancers including Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukaemia (CLL), Myelodysplastic Syndrome (MDS), and Diffuse Large B cell Lymphoma (DLBCL); or Hepatocellular Carcinoma; or Gynaecological Cancers including Ovarian Cancer, Cervical Cancer, and Endometrial Cancer; lung cancer, bladder cancer, breast cancer, renal cell carcinoma, melanoma, colorectal cancer, gastric cancer, pancreatic cancer, thyroid cancer, liver cancer and glioblastoma.

34. A method of targeting cells that express both CD33 and B7H4 comprising administering to a subject an agent that binds to CD33 and B7H4.

35. The method of claim 34 wherein the agent is a bispecific antibody or antigen binding fragment thereof that binds CD33 and B7H4 linked to a cell killing portion.

36. A combination of agents for use in the treatment of a malignancy, wherein the cell inhibiting agents bind to CD33 and B7H4.

37. The combination for use according to claim 36, wherein the combination is non-immune suppressing.

38. The combination for use according to claim 37, wherein the non-immune suppressing treatment is non-myelosuppressing.

39. The combination for use according to any of claims 36 to 38, wherein the agents comprise antibodies or antigen binding fragment thereof.

40. The combination for use according to any of claims 36 to 39, wherein the agents further comprises a payload.

41. The combination for use according to claim 40 wherein the payload is a cell killing agent, an immune-modulating payload, a macrophage class switching agent or a light activatable payload.

42. The combination for use according to claim 41 wherein the immune-modulating payload is a STING agonist or a toll-like receptor agonist.

43. The combination for use according to claim 42, wherein the cell killing agent comprises a cytotoxin.

44. The combination for use according to claim 43, wherein said cytotoxin is selected from:
i) a peptide toxin; or
ii) a chemical toxin.

45. The combination for use according to claims 36 to 46, wherein the agents further comprises a linker for linking the payload to the agent that binds to CD33 and B7H4 expressed on the cell surface.

46. The combination for use according to claims 36 to 39, wherein the agent is an antibody drug conjugate.

47. The combination for use according to claims 32 to 46, wherein the malignancy comprises a malignant disease associated with an increase in myeloid derived suppressor cells.

48. The combination for use according to claims 36 to 47, wherein the malignancy is an ovarian cancer, ovarian cancer derived cancer, AML or AML derived cancer.

49. The combination for use according to claims 36 to 48, wherein the malignancy is selected from one of the following cancers: haematological cancers including Multiple Myeloma (MM), Acute Myeloid Leukaemia (AML), T-Cell Acute Lymphoblastic Leukaemia (T-ALL), Blastic Plasmacytoid Dendritic Cell Neoplasm (BPDCN), Chronic Lymphocytic Leukaemia (CLL), Myelodysplastic Syndrome (MDS) and Diffuse Large B cell Lymphoma (DLBCL); or Hepatocellular Carcinoma; or Gynaecological Cancers including Ovarian Cancer, Cervical Cancer and Endometrial Cancer; lung cancer, bladder cancer breast cancer, renal cell carcinoma, melanoma, colorectal cancer, gastric cancer, pancreatic cancer, thyroid cancer, liver cancer and glioblastoma.

50. A bispecific antibody or antigen binding fragment thereof capable of binding CD33 and B7H4.

51. The bispecific antibody or antigen binding fragment thereof according to claim 50 linked to a payload.

52. The composition for use according to claim 51 wherein the payload is a cell killing agent, an immune-modulating payload, a macrophage class switching agent, or a light activatable payload.

53. The composition for use according to claim 52 wherein the immune-modulating payload is a STING agonist or a toll-like receptor agonist.

54. The bispecific antibody or antigen binding fragment thereof according to claim 52, wherein the payload comprises a cytotoxin.

55. The bispecific antibody or antigen binding fragment thereof according to claim 54, wherein said cytotoxin is selected from:
i) a peptide toxin; or
ii) a chemical toxin.

56. A pharmaceutical composition comprising an antibody or antigen binding fragment thereof according to any of claims 50 to 55.

57. A kit comprising an antibody or antigen binding fragment thereof according to any of claims 50 to 55 or a pharmaceutical composition according to claim 56.


SEQUENCE LISTING (11 sequences)

SEQ ID NO: 1 - protein, 364 aa, Homo sapiens
MPLLLLLPLLWAGALAMDPNFWLQVQESVTVQEGLCVLVPCTFFHPIPYYDKNSPVHGYWFREGAIISGDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 2 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 3 - protein, 116 aa, Homo sapiens [sequence omitted]

SEQ ID NO: 4 - protein, 111 aa, Homo sapiens
DIQLTQSPSTLSASVGDRVTIICRASESLDNYGIRFLTWFQQKPGKAPKLLMYAASNQGSGVPSRFSGSGSGTEFTLTISSLQPDDFATYYCQQTKEVPWSFGQGTKLEIK

SEQ ID NO: 5 - protein, 112 aa, Homo sapiens
DIVMTQTPLSLPVTLGQPASISCRSSQSLVHINGNTYLHWYQQRPGQSPRVLIYKVSNRFSGVPDRFSGSGSGTDFTLKISRVEAEDVGVYFCSQSTHVPLTFGQGTKVEIK

SEQ ID NO: 6 - protein, 115 aa, Homo sapiens
EVQLVQSGAEVKKPGASVKVSCKASGYTFTDYYMNWVRQAPGQGLEWIGIINPYNGDTSYNQKFKGRVTLTVDKSTSTAYMELSSLRSEDTAVYYCARYPESTYWGQGTLVTVSS

SEQ ID NO: 7 - protein, 116 aa, Homo sapiens [sequence omitted]

SEQ ID NO: 8 - protein, 111 aa, Homo sapiens; identical to SEQ ID NO: 4

SEQ ID NO: 9 - protein, 247 aa, Homo sapiens
DIVMTQTPLSLPVTLGQPASISCRSSQSLVHINGNTYLHWYQQRPGQSPRVLIYKVSNRFSGVPDRFSGSGSGTDFTLKISRVEAEDVGVYFCSQSTHVPLTFGQGTKVEIKGGGGSGGGGSGGGGSGGGGSEVQLVQSGAEVKKPGASVKVSCKASGYTFTDYYMNWVRQAPGQGLEWIGIINPYNGDTSYNQKFKGRVTLTVDKSTSTAYMELSSLRSEDTAVYYCARYPESTYWGQGTLVTVSS

SEQ ID NO: 10 - protein, 328 aa, Homo sapiens
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 237 aa, Homo sapiens
GGGGSEPKSQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK